(19) United States
(12) Patent Application Publication
(10) Pub. No.: US 2019/0111130 A1
(43) Pub. Date: Apr. 18, 2019

(54) Compositions and Methods to Enhance Ultrasound-Mediated Delivery of Pharmaceutical Agents

(71) Applicants: Massachusetts Institute of Technology, Cambridge, MA (US); The Brigham and Women's Hospital, Inc., Boston, MA (US)

(72) Inventors: Robert S. Langer, Newton, MA (US); Carlo Giovanni Traverso, Newton, MA (US); Dan Minahan, Weymouth, MA (US); Taylor A. Bensel, Boston, MA (US); Thomas von Erlach, Somerville, MA (US); Carl Magnus Schoellhammer, Medford, MA (US)

(21) Appl. No.: 16/162,060

(22) Filed: Oct. 16, 2018

Related U.S. Application Data
(60) Provisional application No. 62/573,000, filed on Oct. 16, 2017.

(51) Int. Cl. A61K 41/00; A61K 9/00; A61P 1/00; G01N 1/28; A61M 37/00

(52) U.S. Cl. CPC A61K 41/00; A61K 9/0031; A61K 9/0053; A61F 7/0085; G01N 1/286; A61M 37/0092; A61M 2037/0007; A61P 1/00

(57) ABSTRACT

Disclosed herein are compositions comprising a pharmaceutical agent and an ultrasound enhancing agent. The compositions are useful in combination with ultrasound to enhance delivery of a pharmaceutical agent to, for example, tissue of a subject in need thereof. Accordingly, also provided herein are methods involving ultrasound for delivering a pharmaceutical agent to a subject, e.g., a subject that has inflammatory bowel disease or proctitis.

### RELATED APPLICATION

This application claims the benefit of U.S. Provisional Application No. 62/573,000, filed on Oct. 16, 2017, the teachings of which are incorporated herein by reference in their entirety.

### GOVERNMENT SUPPORT

This invention was made with government support under Grant No. R37 EB000244 awarded by the National Institutes of Health. The government has certain rights in the invention.

### BACKGROUND

Ultrasound is broadly used clinically, from imaging to lithotripsy. More recently, ultrasound has been utilized for drug delivery through the skin and gastrointestinal (GI) tract. Nonetheless, there remains a need for further enhancements to ultrasound-assisted drug delivery, especially to reduce treatment time and enhance tissue penetration and dosage control.

### SUMMARY

The present invention is based, at least in part, on the discovery of excipients, dopants and other compounds that interact with ultrasound to enhance the delivery of material to, for example, skin and GI tissue, utilizing short treatment times.

Provided herein is a composition (e.g., pharmaceutical composition) comprising a pharmaceutical agent (e.g., therapeutic agent, diagnostic agent) and an ultrasound enhancing agent (e.g., an agent that enhances cavitational activity in a fluid comprising the pharmaceutical agent; an excipient, such as a disulfide bond-forming agent; a dopant). The ultrasound enhancing agent can be an excipient at a concentration of at least about 1 mg/mL or a dopant at a concentration of at least about 0.05% weight/volume.

Also provided herein is a composition (e.g., pharmaceutical composition) comprising a pharmaceutical agent (e.g., therapeutic agent, diagnostic agent), a first ultrasound enhancing agent (e.g., an excipient, such as a disulfide bond-forming agent) and a second ultrasound enhancing agent (e.g., a dopant; an agent that enhances cavitational activity in a fluid comprising the pharmaceutical agent).

Further provided herein is a method of delivering a pharmaceutical agent to (e.g., tissue of) a subject (e.g., subject in need thereof). The method comprises administering a composition described herein (e.g., an effective amount of a composition described herein) to a region of a subject and delivering ultrasound to the region, thereby delivering the pharmaceutical agent to the subject.

Further provided herein is a method of delivering a pharmaceutical agent to (e.g., tissue of) a subject (e.g., subject in need thereof). The method comprises administering a fluid (e.g., liquid) composition described herein (e.g., an effective amount of a fluid composition described herein) to the subject and delivering ultrasound to the fluid, thereby delivering the pharmaceutical agent to the tissue of the subject.

Yet further provided herein is a method of delivering a pharmaceutical agent to (e.g., tissue of) a subject (e.g., subject in need thereof) comprising administering a pharmaceutical agent (e.g., an effective amount of a pharmaceutical agent) and an ultrasound enhancing agent in one or more fluids (e.g., liquids) to the subject and delivering ultrasound to the one or more fluids. Delivery of the pharmaceutical agent to the tissue of the subject is thereby achieved (e.g., enhanced).

Further provided herein is a method of obtaining a biological sample from a subject. The method comprises delivering a plurality of frequencies of ultrasound to a region, tissue or a portion of tissue of the subject, and extracting the biological sample (e.g., interstitial fluid) from the region, the tissue or the portion of the tissue, thereby obtaining a biological sample from the subject.

Further provided herein is a method of achieving a predetermined permeability of a region, tissue or a portion of tissue of a subject. The method comprises selecting a plurality of frequencies of ultrasound to be delivered to the region, the tissue or the portion of tissue and calculating a time period for delivery of the plurality of frequencies of ultrasound based on the plurality of frequencies selected and the predetermined permeability. The plurality of frequencies of ultrasound is (e.g., then) delivered to the region, the tissue, or the portion thereof, thereby achieving a predetermined permeability of a region, tissue or a portion of tissue of the subject.

Use of the ultrasound enhancing agents and techniques described herein in combination with ultrasound can enhance cavitational activity and increase delivery of material to skin and GI tissue 2-4 times over the delivery that can be achieved using ultrasound alone and an order of magnitude over the delivery that can be achieved using passive diffusion.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments.

FIG. 1A is a bar graph, and shows the amount of fluorescently-labeled latex beads of different sizes delivered into porcine colonic tissue ex vivo compared to delivery without ultrasound (control). Data represent averages +1 standard deviation (SD). Sample size (n) indicates biological repeats.

FIG. 1B is a bar graph, and shows the amount of fluorescently-labeled dextran particles of different sizes delivered into porcine colonic tissue ex vivo compared to delivery without ultrasound (control). Data represent averages +1 standard deviation (SD). Sample size (n) indicates biological repeats.

FIG. 2A is a scanning electron microscopy (SEM) micrograph, and shows porcine colonic tissue not treated with ultrasound.

FIG. 2B is a SEM micrograph, and shows porcine colonic tissue after treatment with ultrasound.

FIG. 2C is a SEM micrograph, and shows porcine colonic tissue after simultaneous treatment with ultrasound and 15-μm diameter latex beads.

FIG. 3A is a z-stack confocal image taken at a tissue depth of 25 μm, and shows porcine colonic tissue after delivery of 0.5-μm diameter carboxylate-modified latex beads and staining with 4′,6-diamidino-2-phenylindole (DAPI). The latex particles are shown in red, DAPI nuclear stain is shown in blue and second harmonics representing the tissue architecture are shown in white.

FIG. 3B is a z-stack confocal image taken at a tissue depth of 50 μm, and shows porcine colonic tissue after delivery of 0.5-μm diameter carboxylate-modified latex beads and staining with DAPI. The latex particles are shown in red, DAPI nuclear stain is shown in blue and second harmonics representing the tissue architecture are shown in white.

FIG. 3C is a z-stack confocal image taken at a tissue depth of 75 μm, and shows porcine colonic tissue after delivery of 0.5-μm diameter carboxylate-modified latex beads and staining with DAPI. The latex particles are shown in red, DAPI nuclear stain is shown in blue and second harmonics representing the tissue architecture are shown in white.

FIG. 3D is a z-stack confocal image taken at a tissue depth of 100 μm, and shows porcine colonic tissue after delivery of 0.5-μm diameter carboxylate-modified latex beads and staining with DAPI. The latex particles are shown in red, DAPI nuclear stain is shown in blue and second harmonics representing the tissue architecture are shown in white.

FIG. 3E is a z-stack confocal image taken at a tissue depth of 125 μm, and shows porcine colonic tissue after delivery of 0.5-μm diameter carboxylate-modified latex beads and staining with DAPI. The latex particles are shown in red, DAPI nuclear stain is shown in blue and second harmonics representing the tissue architecture are shown in white.

FIG. 4 is a bar graph, and shows the amount of 0.2-μm diameter fluorescently labeled latex beads with different surface modifications delivered into porcine colonic tissue ex vivo. Amine-modified beads are cationic and carboxylate-modified beads are anionic. Data represent averages +1SD. P>0.1 by Student's two-tailed t-test. Sample size (n) indicates biological repeats.

FIG. 5A is a line graph, and shows the amount of fluorescently labeled permeant delivered into porcine colonic tissue ex vivo versus ultrasound treatment time for 70 kDa dextran. Data represent averages ±1SD. * indicates P<0.05 by one-way ANOVA with multiple comparisons. ** represents P<0.05 compared to all other treatment times. Each condition represents 3-12 biological repeats.

FIG. 5B is a line graph, and shows the amount of fluorescently labeled permeant delivered into porcine colonic tissue ex vivo versus ultrasound treatment time for 2,000 kDa kDa dextran. Data represent averages ±1SD. * indicates P<0.05 by one-way ANOVA with multiple comparisons. ** represents P<0.05 compared to all other treatment times. Each condition represents 3-12 biological repeats.

FIG. 5C is a line graph, and shows the amount of fluorescently labeled permeant delivered into porcine colonic tissue ex vivo versus ultrasound treatment time for 0.5-nm diameter carboxylate-modified latex beads. Data represent averages ±1SD. * indicates P <0.05 by one-way ANOVA with multiple comparisons. ** represents P<0.05 compared to all other treatment times. Each condition represents 3-12 biological repeats.

FIG. 6A is a bar graph, and shows the amount of fluorescently labeled permeant delivered into porcine colonic tissue ex vivo with and without SLS for 70 kDa dextran. Data represent averages +1SD. ** indicates P<0.05 by two-tailed Student's t-tests. Sample size (n) indicates biological repeats.

FIG. 6B is a bar graph, and shows the amount of fluorescently labeled permeant delivered into porcine colonic tissue ex vivo with and without SLS for 2,000 kDa dextran. Data represent averages +1SD. ** indicates P<0.05 by two-tailed Student's t-tests. Sample size (n) indicates biological repeats.

FIG. 6C is a bar graph, and shows the amount of fluorescently labeled permeant delivered into porcine colonic tissue ex vivo with and without SLS for 0.5-nm diameter carboxylate-modified latex beads. Data represent averages +1SD. ** indicates P <0.05 by two-tailed Student's t-tests. Sample size (n) indicates biological repeats.

FIG. 6D is a bar graph, and shows the fraction of the initial amount of permeant delivered into tissue remaining in the tissue after 24-hour clearance studies for 70 kDa dextran. The amount of 70 kDa dextran is shown after 24 hours normalized to its initial value. Data represent averages +1SD. ** indicates P<0.05 by two-tailed Student's t-tests. Sample size (n) indicates biological repeats.

FIG. 6E is a bar graph, and shows the fraction of the initial amount of permeant delivered into tissue remaining in the tissue after 24-hour clearance studies for 2,000 kDa dextran. The amount of 2,000 kDa dextran is shown after 24 hours normalized to its initial value. Data represent averages +1SD. ** indicates P<0.05 by two-tailed Student's t-tests. Sample size (n) indicates biological repeats.

FIG. 6F is a bar graph, and shows the fraction of the initial amount of permeant delivered into tissue remaining in the tissue after 24-hour clearance studies for 0.5-μm diameter carboxylate-modified latex beads. The amount of 0.5-μm diameter carboxylate-modified latex beads dextran is shown after 24 hours normalized to its initial value. Data represent averages +1SD. ** indicates P<0.05 by two-tailed Student's t-tests. Sample size (n) indicates biological repeats.

FIG. 7A shows a miniaturized 40-kHz ultrasound probe for local administration in mice used in certain examples described in the Exemplification. The protrusions initiate radial ultrasound activity.

FIG. 7B is a graph, and shows the fraction of the initial amount of the indicated permeant delivered into mouse colonic tissue in vivo 30 minutes after administration. Data represents averages +1SD. ** Represents P <0.05 compared to the amount of each permeant delivered into tissue immediately after treatment by a two-tailed Student's t-test. Sample size (n) indicates biological repeats.

FIG. 8A is a diagram, and shows an ultrasound device configured for rectal drug administration of an ultrasound-transmitting chemical formulation, such as a composition described herein.

FIG. 8B is an illustration, and shows that the compositions described herein may be used with a myriad of devices and form factors, including enema-based delivery, lollipop-like systems, and fully ingestible, ultrasound-emitting devices, for use throughout the GI tract.

FIG. 8C is a diagram, and shows the positioning of low- and high-frequency ultrasound horns relative to the tissue surface to be treated in one embodiment of dual-frequency ultrasound. The high-frequency horn projects such that nucleated bubbles may cross the ultrasound field emitted by the low-frequency horn.

FIG. 9A is a diagram of one embodiment of a methodological setup described herein and a cross-section of the setup. The setup allows for high-throughput screening of material for ultrasound-mediated delivery, and includes a custom well plate-like setup creating 12 or more discrete diffusion chambers. The cross-section is shown with tissue mounted between the donor chamber (top) and receiver chamber (bottom).

FIG. 9B is an angled cross-sectional view of the setup illustrated in FIG. 9A.

FIG. 9C is a diagram of one embodiment of a methodological setup described herein, and shows the setup illustrated in FIG. 9A and a multi-element ultrasound probe allowing for discrete sonication of each individual diffusion chamber.

FIG. 10 is a representative image, and shows porcine tissue imaged using a fluorescent imager. The tissue is visible in a petri dish. The 12 discrete spots correspond to the 12 individual wells in the methodological setup depicted in FIGS. 9A-9C, which was used to conduct the experiment leading to this image.

FIG. 11 is a bar graph, and shows enhancement of delivery, defined as the fluorescence intensity of dextran in tissue of a chemical formulation containing the indicated compound, normalized by the intensity achieved using dextran in PBS alone, in colon tissue.

FIG. 12 is a bar graph, and shows the results of a chemical formulation screen for the enhancement in delivery of oxytocin to colon tissue. Formulations showing significant enhancement are highlighted in red. Those showing moderate enhancement are shown in yellow. Oxytocin in PBS alone (the control) is shown in blue.

FIGS. 13A-13F are graphs, and show the effect of pit radius, number of pits and total pitted area in aluminum foil samples by latex bead size and latex bead weight percent in coupling solution without SLS.

FIGS. 13G-13L are graphs, and show the effect of pit radius, number of pits and total pitted area in aluminum foil samples by latex bead size and latex bead weight percent in coupling solution with SLS.

FIGS. 13M-13R are graphs, and show the effect of pit radius, number of pits and total pitted area in aluminum foil samples by silica particle size and silica particle weight percent in coupling solution without SLS.

FIGS. 13S-13X are graphs, and show the effect of pit radius, number of pits and total pitted area in aluminum foil samples by silica particle size and silica particle weight percent in coupling solution with SLS.

FIG. 14 is a bar graph, and shows the current of skin after various treatment regimens.

FIG. 15A is a graph, and shows skin permeability versus localized transport region (LTR) area for skin samples treated with single or dual-frequency ultrasound for 6 minutes.

FIG. 15B is a graph, and shows skin permeability versus localized transport region (LTR) area for skin samples treated with single or dual-frequency ultrasound for 8 minutes.

### DETAILED DESCRIPTION

A description of example embodiments follows.

The teachings of all patents, published applications and references cited herein are incorporated by reference in their entirety. Compositions

Provided herein is a composition (e.g., a pharmaceutical composition) comprising a pharmaceutical agent (e.g., an effective amount of a pharmaceutical agent) and an ultrasound enhancing agent.

When introducing elements disclosed herein, “a,” “an,” “the” and “said” are intended to mean that there are one or more of the elements. Thus, “an ultrasound enhancing agent” includes one ultrasound enhancing agent and a plurality of (e.g., 2, 3, 4, 5, 7, 8, 9, 10) ultrasound enhancing agents. Further, the plurality can comprise more than one of the same ultrasound enhancing agent or a plurality of different ultrasound enhancing agents.

As used herein, “pharmaceutical agent” includes therapeutic agents and diagnostic agents. A “pharmaceutical agent” can be a small molecule (e.g., organic small molecule, inorganic small molecule), polymer (e.g., organic polymer), nucleic acid and/or peptide (e.g., protein). Examples of pharmaceutical peptides include, but are not limited to, oxytocin, insulin, erythropoietin and interferon. Examples of pharmaceutical nucleic acids include, but are not limited to, antisense nucleic acids, genes encoding therapeutic proteins and aptamers. Examples of pharmaceutical small molecules include, but are not limited to, anti-inflammatories, antivirals, antifungals, antibiotics, local anesthetics and saccharides.

Thus, in some embodiments, the pharmaceutical agent is a therapeutic agent. As used herein, “therapeutic agent” refers to a bioactive agent. A “therapeutic agent” can be a small molecule (e.g., organic small molecule, inorganic small molecule), polymer (e.g., organic polymer), nucleic acid and/or peptide (e.g., protein). Examples of therapeutic peptides include, but are not limited to, oxytocin, insulin (for diabetes, for example), erythropoietin and interferon.

Examples of therapeutic nucleic acids include, but are not limited to, antisense nucleic acids, genes encoding therapeutic proteins and aptamers. Examples of therapeutic small molecules include, but are not limited to, steroids (for inflammatory conditions, such as eosinophilic esophagitis, Celiac disease or dermatitis, for example), anti-fibrinolytics (e.g., transexamic acid (for blood loss, for example)), anti-inflammatories (e.g., for psoriasis, 5-aminosalicylate (for Crohn's disease, ulcerative colitis, for example)), irritants (e.g., salicyclic acid (for warts, for example)), antivirals, antifungals, antibiotics, local anesthetics and saccharides. Therapeutic agents include, but are not limited to, drugs (e.g., medicinal drugs, biologics), cosmetics, vaccines and nutraceuticals that are bioactive. In one embodiment, the therapeutic agent is a drug (e.g., a medicinal drug, a biologic).

Therapeutic agents include any known bioactive agents, for example, proteins or peptides such as insulin, erythropoietin and interferon. Other bioactive agents include nucleic acids such as antisense nucleic acids and genes encoding therapeutic proteins, pharmaceutical agents such as synthetic organic and inorganic molecules including anti-inflammatories, antivirals, antifungals, antibiotics, local anesthetics, and saccharides, etc. In certain embodiments, the pharmaceutical agent is a contrast agent. In one embodiment, the pharmaceutical agent is oxytocin.

In other embodiments, the pharmaceutical agent is a diagnostic agent. As used herein, “diagnostic agent” refers to an agent used to examine a subject in order to diagnose a disease in the subject or detect impairment of normal functions in the subject. Diagnostic agents include contrast agents (e.g., x-ray contrast agents), organ function diagnosis agents and radioactive agents. A “diagnostic agent” can be a small molecule (e.g., organic small molecule, inorganic small molecule), polymer (e.g., organic polymer), nucleic acid and/or peptide (e.g., protein). Examples of diagnostic small molecules include, but are not limited to, Congo red, indocyanine green, fluorescein (e.g., fluorescein sodium), barium sulfate or diatriazoic acid.

The amount of the pharmaceutical agent in the composition may be from about 0.1 mg/mL to about 100 mg/mL. For example, the amount of the pharmaceutical agent may be about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 mg/mL. In one embodiment, the pharmaceutical agent is present at a concentration of about 10 mg/mL. In one embodiment, the pharmaceutical agent is present at a concentration of about 10 mg/mL in combination with fluorescently labeled dextran.

In some embodiments, the composition comprises an effective amount (e.g., a therapeutically effective amount, diagnostically effective amount) of the pharmaceutical agent (e.g., therapeutic agent, diagnostic agent). As used herein, an “effective amount” is an amount of an agent that, when administered to a subject, is sufficient to achieve a desired therapeutic or diagnostic effect in the subject under the conditions of administration. The effectiveness of a therapy or diagnostic can be determined by any suitable method known to those of skill in the art (e.g., in situ immunohistochemistry, imaging (e.g., ultrasound, CT scan, MM, NMR, ³H-thymidine incorporation). Determination of an “effective amount” is within the skill of a clinician of ordinary skill using the guidance provided herein and other methods known in the art, and is dependent on several factors including, for example, the particular agent chosen, the subject's age, sensitivity, tolerance to drugs and overall well-being. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per administration. Determining the dosage for a particular agent, subject and disease is well within the abilities of one of skill in the art. Preferably, the dosage does not cause or produces minimal adverse side effects (e.g., immunogenic response, nausea, dizziness, gastric upset, hyperviscosity syndromes, congestive heart failure, stroke, pulmonary edema).

As used herein, “ultrasound enhancing agent” refers to any pharmaceutically acceptable agent that, when administered in combination with ultrasound, enhances, under at least one set of conditions, the delivery of a pharmaceutical agent into a tissue, or a portion thereof, of a subject as compared to an otherwise identical composition including the pharmaceutical agent and lacking the ultrasound enhancing agent. Typically, an ultrasound enhancing agent is applied at a concentration that increases the amount and/or rate of absorption of a pharmaceutical agent into the subject's tissue(s).

Delivery of a pharmaceutical agent into a tissue, or a portion thereof, of a subject is enhanced (e.g., improved, increased) herein when the cavitational activity of a fluid containing a pharmaceutical agent (cavitation activity being indicated by the intensity and/or number of transient cavitation events observed, for example) is enhanced, or when the amount and/or rate of absorption and/or penetration of the pharmaceutical agent into a subject (e.g., a subject's tissue) is enhanced. Cavitational activity can be assessed with aluminum foil pitting experiments, in accordance with the examples provided herein, or by acoustic measurements of sub-harmonics using a hydrophone. Amount of absorption can be assessed, in accordance with the examples provided herein, using in vivo fluorescence-based imaging. Rate of absorption can be assessed, for example, with timed diffusion experiments using a fluorescently-labeled agent, a radiolabeled agent or similar agent. Penetration can be assessed, in accordance with the examples provided herein, by examining localized transport regions, for example, using in vivo fluorescence-based imaging, confocal microscopy or scanning electron microscopy.

In some embodiments, the amount of enhancement of the amount and/or rate of delivery of the pharmaceutical agent is at least 10% or more, up to as high as 300% or more.

In some embodiments, the amount of enhancement of the amount and/or rate of delivery of the pharmaceutical agent is about 1% or more. In some embodiments, the compositions and methods described herein reduce standard deviation, e.g., across experiments, of the amount and/or rate of delivery of the pharmaceutical agent. The reduction of standard deviation is important from a clinical standpoint, where control of dosing is a priority.

It was surprisingly discovered that the methods and compositions described herein were able to provide a remarkable increase in penetration. For example, the penetration enhancement relative to a control may be from about 1% to about 500%. For example, the penetration enhancement using the compositions and methods described herein may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500% enhancement in penetration.

In certain embodiments, the penetration of the composition is to a tissue in the body. In one embodiment, the penetration of the composition is to any epidermal tissue in the body.

Unless otherwise indicated, “penetration” refers to the amount of pharmaceutical agent that penetrates tissue of a subject. When so indicated, “penetration” can also refer to the depth to which a pharmaceutical agent penetrates tissue of a subject, the area of tissue penetrated by a pharmaceutical agent (e.g., the area of the localized transport region) or the rate of penetration of the pharmaceutical agent into tissue of a subject. Penetration of a pharmaceutical agent into tissue of a subject can be measured as described in the Exemplification herein.

In one embodiment, an ultrasound enhancing agent enhances cavitational activity in a fluid comprising a pharmaceutical agent (e.g., a fluid composition described herein). Without wishing to be bound by any particular theory, it is believed that an ultrasound enhancing agent enhances delivery of a pharmaceutical agent into tissue of a subject by enhancing cavitational activity in a fluid comprising the pharmaceutical agent.

As used herein, “fluid composition” refers to a composition described herein in fluid form. Typically, a fluid composition is in liquid form (i.e., is a liquid composition). Fluid compositions include solutions and suspensions. Thus, fluid compositions may include solid(s) in addition to liquid(s) and/or gas(es), though the characteristics of a fluid composition are predominantly that of a fluid. Similarly, liquid compositions may include solid(s) and gas(es) in addition to liquid(s), but the characteristics of a liquid composition are predominantly that of a liquid. In one embodiment, the composition described herein is a fluid composition. In one embodiment, the composition is a liquid composition.

Examples of ultrasound enhancing agents include, but are not limited to, disulfide bond-forming agents, ligands, gelating agents (e.g., agar, alginate, alginic acid, carraghenates, gelatin, gums such as gum Arabic, gum guar, gum traganth, locust bean gum, xanthum gum), ion-responsive materials, alcohol dialkyl diesters (e.g., didodecyl 3,3′-thiodipropionate), dicarboxylic acids (e.g., adipic acid), polysaccharides (e.g., starch, cellulose, glycogen, dietary fiber), lipidopreservatives, sweeteners (e.g., aspartame, sucralose, neotame, acesulfame potassium, saccharin, advantame, glycerin), bile acids (e.g., taurocholic acid, glycocholic acid) or dopants. Specific examples of ultrasound enhancing agents include, but are not limited to sodium lauryl sulfate (SLS), 1,2,4,5-benzenetetracarboxylic acid, 3,3′-thiodipropionic acid, adipic acid, alpha-cyclodextrin, didodecyl-3,3′-thiodipropionate, ethylenediaminetetraacetic acid, cysteine, or a salt or hydrate thereof (e.g., L-cysteine hydrochloride monohydrate), saccharin, taurodeoxycholate (e.g., sodium taurodeoxycholate hydrate), thiosulfate (e.g., sodium thiosulfate), glycolate (e.g., sodium glycolate), poly(lactide glycolide) acid, fructose (e.g., D-fructose), mannose (e.g., D(+)-mannose), KOLLIPHOR® EL, mucin, PLURONIC® F-127, glycerin, 8-arm polyethylene glycol (PEG) and MOWIOL®. In one embodiment, the ultrasound enhancing agent (e.g., excipient) is selected from Table 1. In one embodiment, the ultrasound enhancing agent (e.g., excipient) is selected from Table 2.

In one embodiment, the ultrasound enhancing agent is a disulfide bond-forming agent. As used herein, “disulfide bond-forming agent” refers to any agent that is capable, under appropriate conditions (e.g., physiological conditions), of forming a disulfide bond with a thiol functional group. Examples of disulfide bond-forming agents include, but are not limited to, cysteine, coenzyme A and grapefruit mercaptan, or a salt or hydrate of any of the foregoing. In one embodiment, the disulfide bond-forming agent is cysteine, or a salt or hydrate thereof.

In some embodiments, the ultrasound enhancing agent is a ligand. As used herein, “ligand” refers to an ion or molecule attachable to a metal atom by a coordinating bond or a molecule that binds to another molecule. Examples of ligands include, but are not limited to, 1,2,4,5-benzenetetracarboxylic acid and 3,3′-thiodipropione acid, or a salt or hydrate of either of the foregoing.

In some embodiments, the ultrasound enhancing agent is an ion-responsive material. As used herein, “ion-responsive material” refers to a material that responds to ions as a chemical stimuli. In some embodiments, the ion-responsive material is an anion-responsive material. An “anion-responsive material” responds to anions as a chemical stimuli.

In some embodiments, the ultrasound enhancing agent is a lipidopreservative. As used herein, “lipidopreservative” refers to an agent that prevents or delays breakdown of lipids. Examples of lipidopreservatives include, but are not limited to, ethylenediaminetetraacetic acid.

In some embodiments, the ultrasound enhancing agent is a dopant. As used herein, “dopant” refers to a particle that, in combination with ultrasound, modulates (e.g., increases) cavitational activity of a fluid containing a pharmaceutical agent. A dopant can be charged (e.g., cationic, as a carboxylate-modified dopant, or anionic, as an amine-modified dopant) or uncharged and can range dramatically in size. For example, the diameter of a spherical or substantially spherical dopant can be from about 0.01 microns to about 500 microns, from about 0.01 microns to about 10 microns, from about 0.01 microns to about 5 microns, from about 0.01 microns to about 2.5 microns, from about 1 micron to about 5 microns, from about 1 micron to about 250 microns or from about 10 microns to about 150 microns, such as about 0.02 microns, about 0.1 microns, about 0.2 microns, about 0.5 microns, about 1 micron, about 2 microns, about 3 microns, about 4 microns, about 5 microns or about 150 microns. Examples of dopants include, but are not limited to, silica, latex beads and polystyrene microspheres.

In some embodiments, the ultrasound enhancing agent is a non-dopant ultrasound enhancing agent (i.e., an ultrasound enhancing agent that is not a dopant). Non-dopant ultrasound enhancing agents are also referred to herein as “excipients.” Examples of excipients include, but are not limited to, disulfide bond-forming agents, ligands, gelating agents, ion-responsive materials, alcohol dialkyl diesters, dicarboxylic acids, polysaccharides, lipidopreservatives, sweeteners and bile acids. Specific examples of excipients include, but are not limited to, the excipients listed in Tables 1 and 2.

As used herein, “pharmaceutically acceptable” refers to any agent that is, within the scope of sound medical judgment, suitable for use in contact with the tissues of a subject, for example, humans and lower animals, without undue toxicity, irritation, allergic response and the like, and are commensurate with a reasonable benefit/risk ratio.

As used herein, “in combination with,” when referring to administration of a pharmaceutical agent and/or an ultrasound enhancing agent and delivery of ultrasound to a subject, a region of a subject, a tissue of a subject or a portion of a subject's tissue, includes delivery of ultrasound followed by administration of the pharmaceutical agent and/or ultrasound enhancing agent, concurrent delivery of ultrasound and administration of the pharmaceutical agent and/or ultrasound enhancing agent, and administration of the pharmaceutical agent and/or ultrasound enhancing agent followed by delivery of ultrasound. Preferably, administration of the pharmaceutical agent and/or ultrasound enhancing agent follows delivery of ultrasound or delivery of ultrasound and administration of the pharmaceutical agent and/or ultrasound enhancing agent are concurrent (though not necessarily of identical duration). Concurrent delivery of ultrasound and administration of the pharmaceutical agent and/or ultrasound enhancing agent merely implies that there is overlap between the time period during which ultrasound is delivered and the pharmaceutical agent and/or ultrasound enhancing agent is administered, and includes delivery of ultrasound that precedes, but overlaps with, administration of the pharmaceutical agent and/or ultrasound enhancing agent, administration of the pharmaceutical agent and/or ultrasound enhancing agent that precedes, but overlaps with, delivery of ultrasound, and delivery of ultrasound and administration of the pharmaceutical agent and/or ultrasound enhancing agent that begin and/or end at the same or substantially the same time, or any combination of the foregoing.

Typically, the compositions described herein are fluid compositions, especially liquid compositions. Thus, in some embodiments, the ultrasound enhancing agent (e.g., excipient) is present in a concentration greater than about 1 mg/mL, for example, in a concentration of greater than about 1 mg/mL to about 25 mg/mL or greater than about 1 mg/mL to about 10 mg/mL, such as about 2 mg/mL, about 3 mg/mL, about 4 mg/mL, about 5 mg/mL, about 6 mg/mL, about 7 mg/mL, about 8 mg/mL, about 9 mg/mL or about 10 mg/mL. In some embodiments, the ultrasound enhancing agent (e.g., dopant) is present in a concentration of from about 0.05% weight/volume to about 15% weight/volume, for example, from about 0.1% weight/volume to about 10% weight/volume, from about 0.1% weight/volume to about 5% weight/volume or from about 0.5% weight/volume to about 5% weight/volume, such as about 0.1% weight/volume, about 0.2% weight/volume, about 0.3% weight/volume, about 0.4% weight/volume, about 0.5% weight/volume, about 1% weight/volume, about 1.5% weight/volume, about 2% weight/volume, about 2.5% weight/volume, about 3% weight/volume, about 4% weight/volume or about 5% weight/volume.

In some embodiments, the ultrasound enhancing agent is an excipient at a concentration of at least about 1 mg/mL or a dopant at a concentration of at least about 0.05% weight/volume.

Also provided herein is a composition comprising a pharmaceutical agent (e.g., an effective amount of a pharmaceutical agent), a first ultrasound enhancing agent and a second ultrasound enhancing agent.

In one aspect of a composition comprising more than one ultrasound enhancing agent (e.g., first and second ultrasound enhancing agents), the first ultrasound enhancing agent is an excipient, such as a disulfide bond-forming agent (e.g., cysteine, or a salt or hydrate thereof) and the second ultrasound enhancing agent is a dopant. In a composition comprising more than one ultrasound enhancing agent, it is preferable that at least one of the ultrasound enhancing agents enhances cavitational activity in a fluid comprising a pharmaceutical agent (e.g., a fluid composition described herein) as, for example, a dopant can.

Compositions described herein may be administered orally, parenterally (including subcutaneously, intramuscularly, intravenously and intradermally), topically, rectally, nasally, buccally or vaginally. In some embodiments, provided compositions are administrable intravenously and/or intraperitoneally. In some embodiments, the pharmaceutical composition is administrable locally (e.g., via buccal, nasal, rectal or vaginal route). In some embodiments, the pharmaceutical composition is administrable systemically (e.g., by ingestion).

The compositions of the present invention may be administered topically, locally (via buccal, nasal, rectal or vaginal route), or systemically (e.g., peroral route) to a subject (e.g., a human) in need of treatment for a condition or disease, or to otherwise provide a therapeutic effect. In certain embodiments, the composition is administered to epithelial tissues such as the skin, or oral, nasal, or gastrointestinal mucosa. In particular embodiments, the compositions of the present invention can be administered rectally. Such therapeutic effects include, but are not limited to, antimicrobial effects (e.g., antibacterial, antifungal, antiviral, and anti-parasitic effects); anti-inflammation effects including effects in the superficial or deep tissues (e.g., reduction or elimination or soft tissue edema or redness); elimination or reduction of pain, itch or other sensory discomfort; regeneration or healing enhancement of hard tissues (e.g., enhancing growth rate of the nail or regrowth of hair loss due to alopecia) or increase soft tissue volume (e.g., increasing collagen or elastin in the skin or lips); increasing adipocyte metabolism or improving body appearance (e.g., effects on body contour or shape, and cellulite reduction); and increasing circulation of blood or lymphocytes.

The compositions of the present invention may be administered in an appropriate pharmaceutically acceptable carrier having an absorption coefficient similar to water, such as an aqueous gel. Alternatively, a transdermal patch can be used as a carrier. The pharmaceutical agents of the present invention can be administered in a gel, ointment, lotion, suspension, solution or patch, which incorporate any of the foregoing. Accordingly, in one embodiment, the composition further comprises a pharmaceutically acceptable carrier.

Topical application to the lower intestinal tract can be effected in suitable enema formulation. Accordingly, in one embodiment the pharmaceutical composition is an enema.

For other topical applications, the compositions can be formulated in a suitable ointment containing the active component suspended or dissolved in one or more carriers.

Carriers for topical administration of a pharmaceutical agent described herein include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water.

Compositions provided herein can be orally administered in any orally acceptable dosage form including, but not limited to, aqueous suspensions, dispersions and solutions. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

Compositions suitable for buccal administration include lollipop-compatible formulations, wherein the active ingredient is formulated with a carrier such as sugar and acacia, tragacanth, or gelatin and glycerin.

The amount of a pharmaceutical agent described herein that can be combined with a pharmaceutically acceptable carrier to produce a composition in a single dosage form will vary depending upon the subject treated, the particular mode of administration and the activity of the agent employed. Preferably, compositions should be formulated so that a dosage of from about 0.01 mg/kg to about 100 mg/kg body weight/day of the agent can be administered to a subject receiving the composition.

The pharmaceutical agent can also be encapsulated in a delivery device such as a liposome or polymeric nanoparticle, microparticle, microcapsule, or microsphere (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof. The microparticles can have diameters of between 0.0001 and 100 microns, although a diameter of less than 10 microns is preferred. The microparticles can be coated or formed of materials enhancing penetration, such as lipophilic materials or hydrophilic molecules, for example, polyalkylene oxide polymers and conjugates, such as polyethylene glycol. Liposomes are also commercially available. In some embodiments, one or more of the compounds in the pharmaceutical formulation is taken from the list of compounds identified by the U.S. Food and Drug Administration (FDA) as Generally Recognized as Safe (“GRAS”) or contained in the FDA Inactive Ingredient Guide (“IIG”).

### Article of Manufacture, Kits, Devices

Also provided herein is an article of manufacture comprising a composition described herein encapsulated in a cartridge (e.g., that can be loaded into an ultrasound device). In some aspects, the cartridge is disposable.

Also provided herein is a kit comprising a composition described herein and an ultrasound device. In some embodiments, the ultrasound device comprises a transducer capable of emitting ultrasound and a body configured to hold a cartridge containing the composition for delivery to a subject in need thereof. In some embodiments, the composition is contained within a disposable cartridge (e.g., that can be loaded into the ultrasound device).

Also provided herein is a kit comprising a pharmaceutical agent; an ultrasound enhancing agent; one or more fluids; and an ultrasound device. Typically, the one or more fluids has an absorption coefficient similar to water. In some embodiments, the pharmaceutical agent and/or the ultrasound enhancing agent are in the one or more fluids (e.g., one fluid, such as when the pharmaceutical agent and ultrasound enhancing agent are to be administered in a single composition described herein or when the pharmaceutical agent or ultrasound enhancing agent is to be delivered in solid form and the ultrasound enhancing agent or pharmaceutical agent, respectively, is to be delivered (separately) in fluid form; or two, three, four or five fluids, such as when a pharmaceutical agent and ultrasound enhancing agent are both to be administered in fluid form, but in separate compositions from one another). In other embodiments, the pharmaceutical agent and/or the ultrasound enhancing agent are provided separately from the one or more fluids, e.g., for reconstitution prior to administration to a subject.

In some embodiments, the ultrasound device comprises a transducer capable of emitting ultrasound and a body configured to hold a cartridge containing the composition for delivery to a subject in need thereof. The composition and/or the ultrasound enhancing agent and/or the one or more fluids can be contained within a disposable cartridge (e.g., that can be loaded into the ultrasound device).

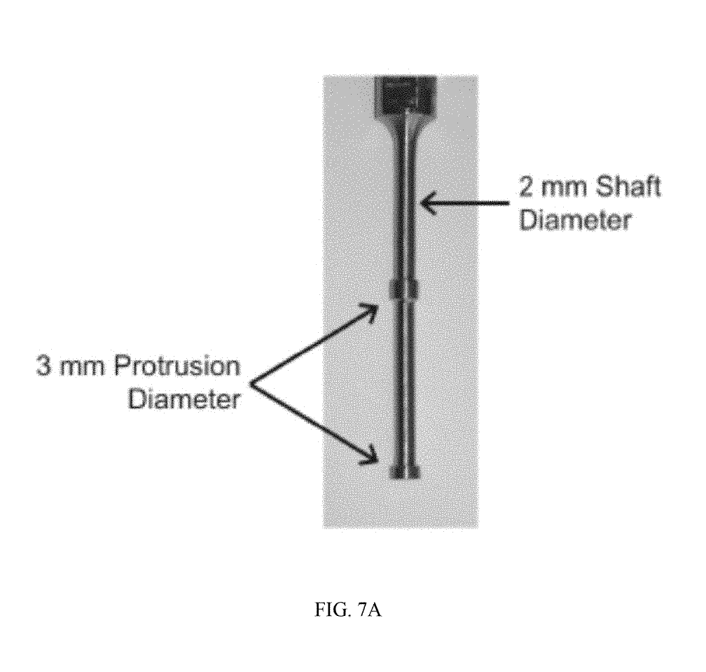

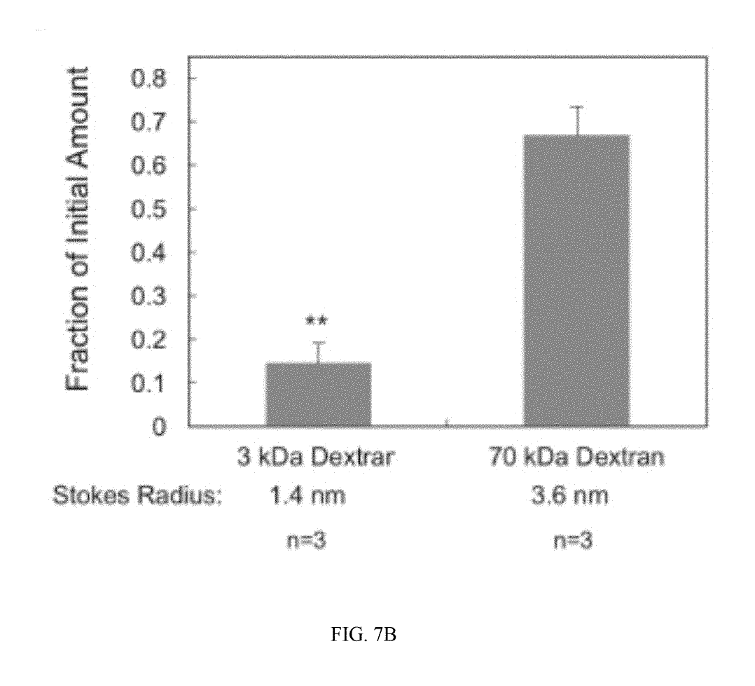

As used herein, “ultrasound device” refers to any device or machine comprising a transducer capable of emitting ultrasound energy (e.g., waves). Ultrasound devices are well-known in the art, and include the ultrasound devices described in International Publication No. WO 2016/164821 as well as the ultrasound devices depicted in FIGS. 7A, 8A-8C and 9A-9C.

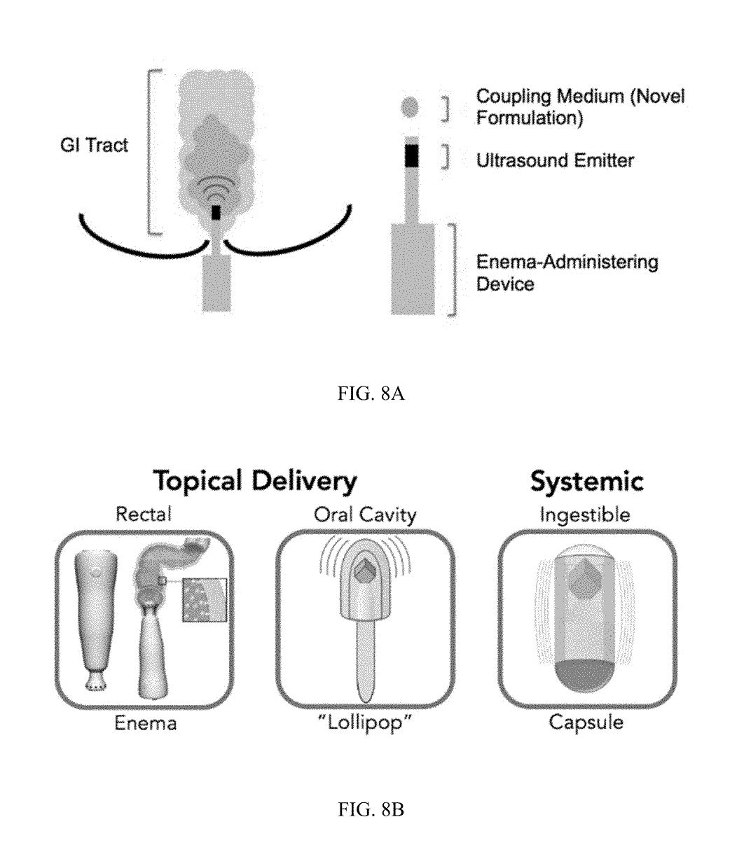

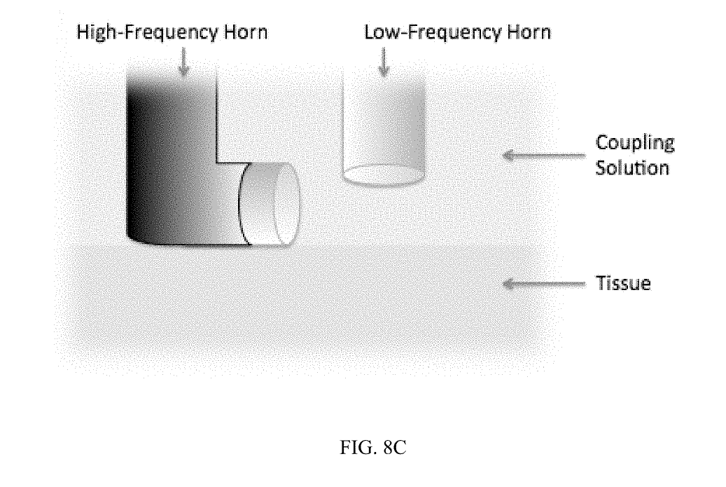

Also provided herein (e.g., for systemic administration of a composition) is an ingestible capsule (e.g., for use in the gastrointestinal tract) comprising a composition described herein and an ultrasound device. One embodiment of such a device is depicted in FIG. 8B, which shows an ingestible, digestible capsule encapsulating an ultrasound device. Also present in the capsule is a composition, such as a composition described herein, formulated to be released, e.g., within the gastrointestinal tract of a subject.

Also provided herein (e.g., for buccal administration of a composition described herein) is a device comprising a composition described herein (e.g., a composition described herein formulated to dissolve in the buccal cavity of a subject) and an ultrasound device configured to be inserted into the buccal cavity of the subject and to deliver ultrasound to the buccal cavity. One embodiment of such a “lollipop” device is depicted in FIG. 8B, which shows an ultrasound device mounted on a handle, the ultrasound device being configured to be inserted into a buccal cavity of a subject and to deliver ultrasound to the buccal cavity. Also provided by the “lollipop” device (e.g., coated on an exterior portion of the device) is a composition, such as a composition described herein, formulated to dissolve in the buccal cavity, for example, upon being licked or sucked on by a subject.

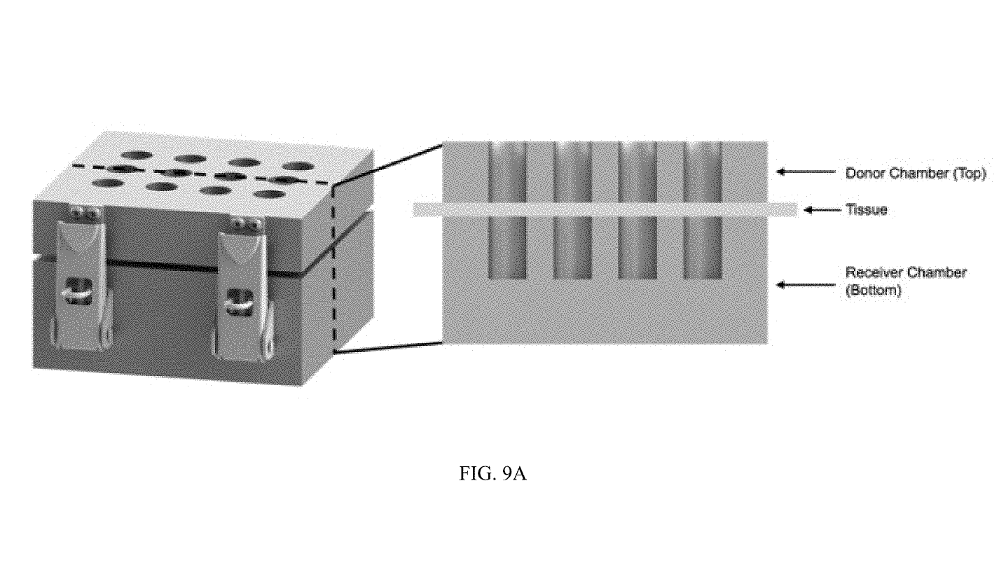

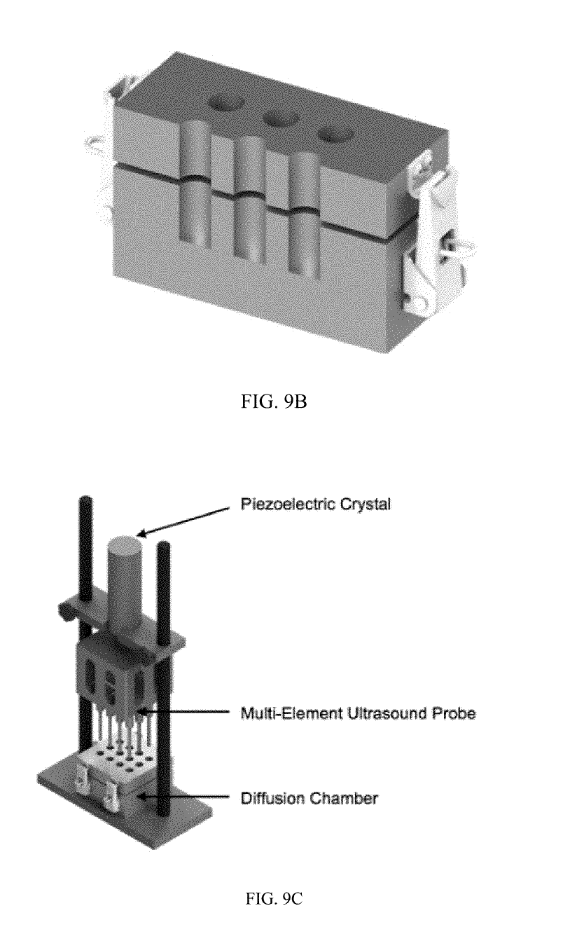

Also provided herein is a well plate (e.g., a multi-well plate composed of from 2 to 100,000 individual wells), comprising a first portion containing one or more (e.g., 1, 2, 6, 12, 36, 72, 96) donor chambers and a second portion containing one or more (e.g., 1, 2, 6, 12, 36, 72, 96) receiver chambers. When the well plate is assembled, each donor chamber is aligned with a receiver chamber so as to form a diffusion chamber, and the first portion and the second portion are configured to receive a tissue sample between them such that the tissue sample is exposed to the contents of each diffusion chamber. In the embodiment of such a well plate depicted in FIGS. 9A-9C, the first portion includes twelve donor chambers, the second portion includes twelve receiver chambers and the first and second portions are secured to one another (with or without a tissue sample mounted between them) by four clamps. It will be appreciated that there are other means of securing the first and second portions to one another, and that such other means are within the scope of this invention.

Also provided is a setup comprising a well plate described herein and an ultrasound device. In some embodiments, the ultrasound device includes a separate ultrasound element for each diffusion chamber in the well plate. An embodiment of such a setup is depicted in FIG. 9C. In other embodiments, the ultrasound device includes a single ultrasound element. Such an embodiment would be particularly useful with a well plate capable of transmitting ultrasound. In use, in such a setup, a tissue sample would be exposed to a single source of ultrasound.

### Methods of Delivery, Treatment

Also provided herein is a method of delivering a pharmaceutical agent to (e.g., tissue of) a subject (e.g., a subject in need thereof). The method comprises administering a composition comprising a pharmaceutical agent described herein (e.g., an effective amount of a composition comprising a pharmaceutical agent described herein) to a region of a subject and delivering ultrasound (e.g., an effective amount of ultrasound) to the region, thereby delivering the pharmaceutical agent to the subject. In one embodiment, the composition is a composition described herein (e.g., an effective amount of a composition described herein).

As used herein, “subject in need thereof” refers to a subject who has, or is at risk for developing, a disease or condition treatable by a therapeutic agent described herein, or diagnosable using a diagnostic agent described herein. A skilled medical professional (e.g., physician) can readily determine whether a subject has, or is at risk for developing, a disease or condition treatable by a therapeutic agent described herein or diagnosable using a diagnostic agent described herein. Examples of subjects in need thereof, include, but are not limited to, mammals (e.g., human, non-human primate, cow, sheep, goat, horse, dog, cat, rabbit, guinea pig, rat, mouse or other bovine, ovine, equine, canine, feline, or rodent organism). In a particular embodiment, the subject is a human.

Ultrasound is a sound wave typically characterized as having a frequency above the audible range of humans (e.g., >20 kHz). Ultrasound has seen broad clinical use for a myriad of applications, including imaging, lithotripsy, and lysis of fat during liposuction. With respect to drug delivery, ultrasound has been investigated for decades for transdermal drug delivery. Without wishing to be bound by any particular theory, it is believed that the enhancement in drug uptake using ultrasound relies on a phenomenon known as acoustic cavitation. When an ultrasound wave is propagating through a fluid, the oscillating pressure field spontaneously nucleates bubbles in the solution. Using low-frequency ultrasound (e.g., less than or equal to 100 kHz), these bubbles grow through rectified diffusion, and eventually become unstable. They then implode, causing a microjet. These microjets can physically propel drug into tissue and reversibly permeabilize tissue to allow enhanced drug uptake.

Ultrasound treatment may be carried out in a variety of methods readily apparent to a skilled artisan. The parameters described herein are not meant to be restrictive, and a skilled artisan will readily appreciate that the parameters may be modified as needed (e.g., to achieve a specified effect). In one embodiment, ultrasound is delivered for a time period of from about 1 minute to about 5 minutes.

Treatment may be carried out for a time period as needed to achieve a therapeutic effect. For example, the treatment may be carried out for a time period from about 1 second to 1 hour. For example, the treatment may be carried out for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 seconds. For example, the treatment may be carried out for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 minutes. In one embodiment, the treatment is carried out for 2 minutes.

In one embodiment, the frequency of the ultrasound is from about 1 kHz to about 100 kHz, about 1 kHz to about 50 kHz or about 20 kHz to about 50 kHz.

The ultrasound frequency may be modified to achieve a particular therapeutic effect. For example, the frequency may be from about 1 kHz to about 1 GHz. For example, the frequency may be about 10, 20, 30, 40, 50, 60, 70, 80, 90, 100, 200, 300, 400, 500, 600, 700, 800, 900, or about 1000 kHz. For example, the frequency may be about 1, 2, 3, 4, 5, 6, 7, 8, 9, 10, 20, 30, 40, 50, 60, 70, 80, 90, 100, 200, 300, 400, 500, 600, 700, 800, 900, to about 1000 MHz. In one embodiment, the frequency is about 20 kHz.

In one embodiment, the intensity of the ultrasound is from about 1 W/cm² to about 10 W/cm².

The ultrasound intensity may be from about 1 W/cm² to about 100 W/cm². For example, the ultrasound intensity may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 W/cm². In one embodiment, the ultrasound intensity is about 5 W/cm².

Ultrasound treatments of the invention include any combination of treatment time, frequency and intensity described herein. For example, in one embodiment, treatment is carried out for 2 minutes total at 50% duty cycle using 20 kHz ultrasound at an intensity of 5 W/cm².

Plural (e.g., dual) frequency ultrasound may also be employed. Ultrasound treatment with plural frequency ultrasound includes treatment with low frequency ultrasound and high frequency ultrasound. Typically, the frequency of the low frequency ultrasound is from about 1 kHz to about 50 kHz, for example, from about 1 kHz to about 25 kHz, from about 10 kHz to about 50 kHz, about 20 kHz or about 25 kHz. The frequency of the high frequency ultrasound is typically from about 500 kHz to about 10,000 kHz, for example, from about 500 kHz to about 5,000 kHz, from about 500 kHz to about 2,500 kHz, from about 500 kHz to about 1,500 kHz, or about 1 MHz.

When dual frequency ultrasound is employed, an ultrasound device configured to deliver low-frequency ultrasound can be positioned so as to emit ultrasound energy (e.g., waves) at an angle perpendicular or substantially perpendicular to the surface of the region or tissue of the subject to which ultrasound is being delivered. An ultrasound device configured to deliver high-frequency ultrasound can be positioned so as to emit ultrasound energy (e.g., waves) parallel or substantially parallel to a surface of the region or tissue of the subject to which ultrasound is being delivered (or at an angle perpendicular or substantially perpendicular to the ultrasound energy (e.g., waves) emitted by the ultrasound device configured to deliver low-frequency ultrasound). One arrangement of ultrasound devices in a dual frequency ultrasound set-up in accordance with this aspect of the invention is depicted in FIG. 8C. FIG. 8C shows a high-frequency ultrasound horn projecting perpendicularly to a low-frequency ultrasound horn, which, in turn, is configured to project ultrasound energy (e.g., waves) at an angle perpendicular or substantially perpendicular to the surface of the region of tissue to which ultrasound is being applied.

The region can include, consist essentially of or consist of any of the organ systems, such as the digestive system (e.g., gastrointestinal tract), the excretory system (e.g., urinary system), the reproductive system, the respiratory system (e.g., during surgery), the nervous system (e.g., during surgery), or an organ thereof (e.g., rectum, vagina, skin), or an anatomical cavity (e.g., peritoneal cavity (e.g., during surgery)), or a portion of any of the foregoing. In one embodiment, the region is the gastrointestinal tract, or a portion thereof. In one embodiment, the region is the subject's skin, or a portion thereof.

The methods described herein can be used to deliver a pharmaceutical agent to a variety of anatomical cavities, including vaginal, urinary system, skin, bronchial/pulmonary system (during surgery), nervous system (during surgery), and peritoneal cavity (during surgery).

In one embodiment, the composition is administered after delivering ultrasound to the region or tissue. In one embodiment, the composition is administered before delivering ultrasound to the region or tissue. Alternatively, administering the composition and delivering ultrasound to the region or tissue are concurrent. Concurrent administration of the composition and delivery of ultrasound includes delivery of ultrasound that precedes, but overlaps with, administration of the composition, administration of the composition that precedes, but overlaps with, delivery of ultrasound and delivery of ultrasound and administration of the composition that begin and/or end at the same time or substantially the same time, or any combination of the foregoing.

In some embodiments, ultrasound is delivered to the subject (e.g., a region or tissue or a portion of a tissue of the subject) before the composition is administered and again upon administration of the composition (either after administration or concurrently with administration). This can increase skin penetration by increasing skin permeability prior to delivery of a pharmaceutical agent. Delivery of ultrasound to the subject prior to administration of the composition is achieved, in some embodiments, using a method of achieving a predetermined tissue permeability described herein.

In one embodiment, the method further comprises freezing the region or tissue of the subject, for example, by exposing the region or tissue to liquid nitrogen. In one aspect, freezing the region or tissue of the subject occurs prior to delivering ultrasound to the region or tissue. Alternatively, freezing and delivering ultrasound are concurrent, wherein concurrent delivery of ultrasound and freezing includes delivery of ultrasound that precedes, but overlaps with, freezing, freezing that precedes, but overlaps with, delivery of ultrasound and delivery of ultrasound and freezing that begin and/or end at the same time or substantially the same time, or any combination of the foregoing.

Also provided herein is a method of delivering a pharmaceutical agent to (e.g., tissue of) a subject (e.g., a subject in need thereof), comprising administering a fluid composition described herein (e.g., an effective amount of a fluid composition described herein) to the subject and delivering ultrasound (e.g., an effective amount of ultrasound) to the fluid, thereby delivering the pharmaceutical agent to the subject. Variations to this method include those variations described with respect to the method of delivering a pharmaceutical agent to a subject in need thereof comprising administering a composition to a region of a subject.

The tissue of a subject can include, consist essentially of or consist of any of the tissue that makes up an organ system, such as the digestive system (e.g., gastrointestinal tract), the excretory system (e.g., urinary system), the reproductive system, the respiratory system (e.g., during surgery), the nervous system (e.g., during surgery), or the tissue of an organ itself (e.g., rectum, vagina, skin), or tissue of an anatomical cavity (e.g., peritoneal cavity (e.g., during surgery)), or a portion of any of the foregoing. In one embodiment, the tissue is gastrointestinal tissue, or a portion thereof. In one embodiment, the tissue is skin, or a portion thereof.

Also provided herein is a method of delivering a pharmaceutical agent to (e.g., tissue of) a subject (e.g., a subject in need thereof), comprising administering a pharmaceutical agent (e.g., an effective amount of a pharmaceutical agent) and an ultrasound enhancing agent to a region of the subject and delivering ultrasound (e.g., an effective amount of ultrasound) to the region, thereby delivering the pharmaceutical agent to the subject. Variations to this method include those variations described with respect to the method of delivering a pharmaceutical agent to a subject in need thereof comprising administering a composition to a region of a subject as well as the method of delivering a pharmaceutical agent to a subject in need thereof comprising administering a fluid composition to the subject.

Also provided herein is a method of delivering a pharmaceutical agent to (e.g., tissue of) a subject (e.g., a subject in need thereof), comprising administering a pharmaceutical agent (e.g., an effective amount of a pharmaceutical agent) and an ultrasound enhancing agent in one or more fluids to the subject (e.g., one fluid, such as when the pharmaceutical agent and ultrasound enhancing agent are administered in a single composition described herein or when the pharmaceutical agent or ultrasound enhancing agent is delivered in solid form and the ultrasound enhancing agent or pharmaceutical agent, respectively, is delivered (separately) in fluid form; or two, three, four or five fluids, such as when a pharmaceutical agent and ultrasound enhancing agent are both administered in fluid form, but in separate compositions from one another). Ultrasound (e.g., an effective amount of ultrasound) is delivered to the one or more fluids, thereby delivering the pharmaceutical agent to the subject. Variations to this method include those variations described with respect to the method of delivering a pharmaceutical agent to a subject in need thereof comprising administering a composition to a region of a subject as well as the method of delivering a pharmaceutical agent to a subject in need thereof comprising administering a fluid composition to the subject.

When the pharmaceutical agent and ultrasound enhancing agent are administered separately (e.g., in separate compositions), administration of the pharmaceutical agent can occur before, after or concurrently with administration of the ultrasound enhancing agent, so long as the conditions of administration are such that delivery of the pharmaceutical agent to a region or a tissue, or a portion thereof, of a subject is enhanced as compared to delivery of the pharmaceutical agent in the absence of the ultrasound enhancing agent. Concurrent administration of the pharmaceutical agent and the ultrasound enhancing agent includes administration of the pharmaceutical agent that precedes, but overlaps with, administration of the ultrasound enhancing agent, administration of the ultrasound enhancing agent that precedes, but overlaps with, administration of the pharmaceutical agent and administration of the pharmaceutical agent and administration of the ultrasound enhancing agent that begin and/or end at the same time or substantially the same time, or any combination of the foregoing.

In one embodiment, the subject has a disease or condition treatable by a composition or method described herein (e.g., inflammatory bowel disease, proctitis). Diseases and/or conditions treatable using the compositions and methods of the invention include infections (e.g., viral, bacterial, fungal, parasitic), edema (e.g., soft tissue edema), alopecia, warts, psoriasis, infection (e.g., bacterial), dermatitis, inflammatory bowel disease (e.g., Crohn's disease, ulcerative colitis), proctitis (e.g., active proctitis), cystitis (e.g., interstitial cystitis), gastrointestinal bleeding, neoplasia, blood loss, cancer (e.g., vaginal, cervical cancer; peritoneal metastases) and inflammatory conditions of, e.g., the colon, intestine, esophagus, mouth (e.g., eosinophilic esophagitis, eosinophilic enteritis, Celiac disease, oral inflammation). Diseases and/or conditions treatable rectally using the compositions and methods of the invention include inflammatory bowel disease (e.g., Crohn's disease, ulcerative colitis) and proctitis (e.g., active proctitis). Diseases and/or conditions treatable gastrointestinally using the compositions and methods of the invention include gastrointestinal bleeding, neoplasia, blood loss, cancer and inflammatory conditions (e.g., eosinophilic esophagitis, eosinophilic enteritis, Celiac disease). Diseases and/or conditions treatable vaginally using the compositions and methods of the invention include bacterial infection, vaginal cancer and cervical cancer.

Thus, one embodiment is a method of treating a disease or condition in a subject in need thereof, comprising administering to the subject a composition comprising a pharmaceutical agent described herein (e.g., a composition described herein) and delivering ultrasound (e.g., an effective amount of ultrasound) to the subject (e.g., a region of a subject, tissue of a subject or a portion thereof). In one aspect, the disease or condition is inflammatory bowel disease. In one aspect, the disease or condition is proctitis.

As used herein, the terms “treat,” “treating,” or “treatment,” mean to counteract a medical condition to the extent that the medical condition is improved according to a clinically-acceptable standard.

The compositions and methods described herein can be used with particular pharmaceutical agents to treat the following diseases or conditions (with the particular pharmaceutical agent listed in parentheses following the disease or condition): diabetes (insulin); blood loss (transexamic acid); Crohn's disease (5-aminosalicylate); ulcerative colitis (5-aminosalicylate); warts (salicyclic acid).

A pharmaceutical agent can be administered (e.g., formulated with) lidocaine for use during cystoscopy.

The compositions and methods described herein can also be used to administer (or enhance administration of) vaccines. Without wishing to be bound by any particular theory, it is believed that by increasing the permeability of skin, for example, greater quantities of larger antigen particles could be transported through the skin to the underlying cells of the immune system.

The compositions and methods described herein also have cosmetic applications. Thus, the compositions and methods described herein can be used to topically administer skin appearance-modifying agents.

A composition described herein can be administered in a single dose or as multiple doses, for example, in an order and on a schedule suitable to achieve a desired therapeutic or diagnostic effect. Suitable dosages and regimens of administration can be determined by a clinician of ordinary skill.

A composition described herein can also be administered in combination with one or more other therapies or treatments in addition to ultrasound. With respect to the administration of a composition in combination with one or more other therapies or treatments in addition to ultrasound, the composition is typically administered as a single dose (by, e.g., injection, infusion, orally), followed by repeated doses at particular intervals (e.g., one or more hours) if desired or indicated.

When administered in a combination therapy, the composition can be administered before, after or concurrently with the other therapy (e.g., an additional agent(s)). When co-administered concurrently, the composition and other therapy can be in separate formulations or the same formulation. Alternatively, the composition and other therapy can be administered sequentially, as separate compositions, within an appropriate time frame as determined by a skilled clinician (e.g., a time sufficient to allow an overlap of the pharmaceutical effects of the therapies).

The actual dose of a pharmaceutical agent(s) and other therapy(ies) or treatment(s) in a combination treatment regimen can be determined by the physician, taking into account the nature of the disease, other therapies being given, and subject characteristics.

A composition described herein can be administered via a variety of routes of administration, including, for example, oral, dietary, topical, transdermal, rectal, vaginal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the pharmaceutical composition and the particular disease or condition to be treated or diagnosed. Administration can be local or systemic (e.g., local) as indicated. In one embodiment, administration is topical. In one embodiment, administration is local. In one embodiment, administration is oral.

In one embodiment, the composition is administered orally, topically, locally or a combination thereof.

### Methods Involving Plural Frequency Ultrasound

It was unexpectedly discovered that in the presence of plural (e.g., dual) frequency ultrasound, permeability of tissue correlates linearly with the area of localized transport region, which is further correlated to treatment time. This discovery can be exploited in sensing applications to not only detect, but also quantify, a particular biomarker, such as glucose, in a biological sample.

Thus, provided herein is a method of obtaining a biological sample from a subject. The method comprises delivering a plurality of frequencies of ultrasound to a region, tissue or a portion of tissue of the subject, and extracting the biological sample (e.g., a fluid, such as interstitial fluid) from the region, the tissue or the portion of the tissue, thereby obtaining a biological sample from the subject. Typically, extracting occurs after delivery of the plurality of frequencies of ultrasound, although extracting can also occur concurrently with delivery of the plurality of frequencies of ultrasound. Concurrent extraction and delivery of ultrasound includes delivery of ultrasound that precedes, but overlaps with, extraction of the biological sample, extraction of the biological sample that precedes, but overlaps with, delivery of ultrasound, and delivery of ultrasound and extraction of the biological sample that begin and/or end at the same time or substantially the same time, or any combination of the foregoing.

Also provided herein is a method of achieving a predetermined permeability of a region, tissue or a portion of tissue of a subject. The method comprises selecting a plurality of frequencies of ultrasound to be delivered to the region, the tissue or the portion of tissue and calculating a time period for delivery of the plurality of frequencies of ultrasound based on the plurality of frequencies selected and the predetermined permeability. The plurality of frequencies of ultrasound is (e.g., then) delivered to the region, the tissue, or the portion thereof, thereby achieving a predetermined permeability of a region, tissue or a portion of tissue of the subject.

The method can be useful in sensing applications to detect and/or quantify a biomarker in a biological sample. Thus, in some aspects, the method further comprises extracting a biological sample (e.g., a fluid, such as interstitial fluid) from the permeabilized region, tissue or portion of tissue of the subject.

Methods of achieving a predetermined permeability can also be useful in determining treatment time and achieving penetration of a predetermined amount of, for example, an administered dosage of, a pharmaceutical agent, and even identifying pharmaceutical agents deliverable using ultrasound-mediated delivery, for example, by comparison of achievable permeability with the size of a pharmaceutical agent. Thus, in some aspects, the method further comprises administering a pharmaceutical agent (e.g., a composition comprising a pharmaceutical agent, such as a composition described herein) to the permeabilized region, tissue or portion of tissue of the subject.

In some aspects of a method involving plural frequency ultrasound, the plurality of frequencies comprises low frequency ultrasound and high frequency ultrasound. In some aspects, the frequency of the low frequency ultrasound is from about 1 kHz to about 50 kHz. In some aspects, the frequency of the high frequency ultrasound is from about 500 kHz to about 10,000 kHz.

In addition to those variations explicitly described herein, variations to the methods involving plural frequency ultrasound include those variations described with respect to methods of delivery and treatment.

It should also be understood that a method of achieving a predetermined permeability of a region, tissue or a portion of tissue of a subject can be combined with a method of delivery and/or treatment described herein. In particular, in some embodiments, delivery of ultrasound in the methods of delivery and/or treatment described herein includes the method of achieving a predetermined permeability of a region, tissue or a portion of tissue of a subject. Alternatively, in some embodiments, the methods of delivery and/or treatment described herein further include, typically prior to delivery of ultrasound and administration of a pharmaceutical agent or composition described herein, the method of achieving a predetermined permeability of a region, tissue or a portion of tissue of a subject.

### Screening Methods

Also provided herein is a method of identifying a composition for delivery to a subject in combination with ultrasound. The method comprises contacting one or more regions of a test tissue with one or more potential compositions (e.g., one or more compositions of the invention), applying ultrasound to each region of the tissue and examining each region for a property of interest. The presence of the property of interest indicates the presence of a pharmaceutical composition for delivery to a subject in combination with ultrasound.

It will be understood that if presence of a property of interest is indicated by absence of a particular signal, presence of the property of interest is absence of the signal. Conversely, if presence of a property of interest is indicated by presence of a particular signal (e.g., a fluorescent signal), presence of the property of interest is presence of the signal.

Presence of a property can be detected and/or quantified, for example, with in vivo fluorescence imaging, high-performance liquid chromatography (HPLC) (e.g., of receiver chamber fluid) and/or scintillation counting of solubilized tissue sample.

In one embodiment of the method of identifying a composition for delivery to a subject in combination with ultrasound, ultrasound is applied to each region of the tissue individually. One embodiment of such a device is depicted in FIG. 9C. FIG. 9C shows a multi-element ultrasound probe, which allows for discrete sonication of each individual diffusion chamber in the multi-well plate depicted in FIG. 9C.

In one embodiment, ultrasound is applied to each region of the tissue collectively. Ultrasound could be applied to each region of the tissue collectively in a multi-well plate such as that depicted in FIGS. 9A and 9B, for example, if the multi-well plate was capable of transmitting ultrasound.

In one aspect of the method of identifying a composition for delivery to a subject in combination with ultrasound, the method is conducted in a multi-well plate. The multi-well plate comprises a first portion containing multiple donor chambers and a second portion containing multiple receiver chambers. When the multi-well plate is assembled, each donor chamber is aligned with a receiver chamber so as to form a diffusion chamber, and the first portion and the second portion are configured to receive a tissue sample between them such that the tissue sample is exposed to the contents of each diffusion chamber. A representative example of such a device is depicted in FIGS. 9A-9C.

### EXEMPLIFICATION

### Defining Optimal Permeant Characteristics for Ultrasound-mediated Gastrointestinal Delivery

The effect of permeant size, charge and the presence of chemical penetration enhancers on delivery to GI tissue was investigated using ultrasound. Short ultrasound treatments enabled delivery of large permeants, including microparticles, deep into colonic tissue ex vivo. Delivery was relatively independent of size and charge but did depend on conformation, with regular, spherical particles being delivered to a greater extent than long-chain polymers. The subsequent residence time of model permeants in tissue after ultrasound-mediated delivery was found to depend on size, with large microparticles demonstrating negligible clearance from the local tissue 24 hours after delivery ex vivo. The dependence of clearance time on permeant size was further confirmed in vivo in mice using fluorescently labeled 3-kDa and 70-kDa dextran. The use of low-frequency ultrasound in the GI tract represents a tool for the delivery of a wide range of therapeutics independent of formulation, potentially allowing for the tailoring of formulations to impart novel pharmacokinetic profiles once delivered into tissue.

### Materials and Methods.

Chemicals. Phosphate buffered saline (PBS), dextran labeled with Texas red (3 kDa and 70 kDa), dextran labeled with tetramethylrhodamine (2000 kDa), and carboxylate-modified and amine-modified polystyrene microspheres were obtained from Thermo Fisher Scientific (Waltham, Mass.). Sodium hydroxide was obtained from Amresco (Solon, Ohio). Sodium lauryl sulfate (SLS) and formalin were obtained from Sigma-Aldrich (Saint Louise, Mo.). All chemicals were used as received.

Tissue procurement. This research was approved by the Massachusetts Institute of Technology (MIT) Committee on Animal Care. Fresh GI tissue from Yorkshire pigs was procured within an hour of sacrifice. The tissue was washed thoroughly using PBS and excess fat dissected away. The tissue was sectioned into pieces approximately 2 cm×2 cm for subsequent mounting in Franz diffusion cells with an exposed area for delivery of 15 mm (PermeGear, Hellertown, Pa.). First, the receiver chamber was filled with PBS and the tissue placed on top of the receiver chamber with the muscularis layer in contact with the receiver chamber. A donor chamber was then placed on top of the tissue and the setup clamped together. PBS was added to the donor chamber to keep the tissue hydrated before treatment. Care was taken to ensure there were no air bubbles in the receiver chamber. Experiments were conducted at room temperature.

Ultrasound Treatment. Ultrasound was generated with a 20 kHz, VCX500 probe from Sonics & Materials (Newtown, Conn.). Ultrasound was applied with the transducer positioned 3 mm above the tissue surface at an intensity of 5 W/cm² calibrated by calorimetry. A 50% duty cycle was utilized to reduce thermal effects. Immediately before treatment, the PBS was removed from the donor chamber and the coupling fluid containing the permeant of interest was added. Fluorescently labeled probes were used as the model permeants and used at a concentration of 0.2 mg/mL unless otherwise stated.

Delivery quantification in tissue. Permeant content in the tissue after delivery was quantified using an In Vivo Imaging System (IVIS) Fluorescent Imager (PerkinElmer, Waltham, Mass.). Immediately after ultrasound treatment, the donor chamber solution was discarded and the tissue washed. Tissue samples were then imaged with the IVIS Fluorescent Imager. Unless otherwise noted, imaging was performed using a binning factor of 8, f-stop of 8, and a field of view of 21.6 cm. Exposure time was varied to ensure a total photon count of ≥6000, per the manufacturer's guidelines.

Tissue clearance tests. Permeant clearance from tissue samples was also investigated ex vivo. Tissue samples were treated in Franz diffusion cells as described. After treatment, the treated tissue samples were thoroughly washed and placed in individual 500 mL beakers filled with 300 mL PBS to mimic an infinite-sink condition. All beakers were stirred on a magnetic stir plate at 400 rpm. 24 hours after treatment, tissue samples were removed from the beakers, thoroughly washed, and imaged using an IVIS Fluorescent Imager.

Scanning electron microscopy (SEM). In order to image microparticles within tissue after delivery, samples were imaged by scanning electron microscopy using a JEOL JSM-5000 scanning electron microscope and environmental scanning electron microscope. Samples were prepared for imaging by dehydration in 200 proof ethanol at serial concentrations of 50%, 75%, 90%, 95%, and 100% ethanol. Dehydration in each concentration lasted 20 minutes. Ethanol-dehydrated samples were finally dried using a critical point drying instrument. Dried samples were mounted on aluminum stages using carbon black stickers and coated with gold nanoparticles by spattering. Samples were imaged using an acceleration voltage of 5 kV, working distance of 20 mm and a spot size of 20 at various magnifications.

Confocal microscopy. Fluorescently labeled permeants were also imaged for their distribution within tissue by confocal microscopy. After ultrasound treatment, the tissue was thoroughly washed and removed from the Franz diffusion cells. Tissue was fixed with 10% formalin overnight. After fixation, tissue samples were stained with 4′,6-diamidino-2-phenylindole, dihydrochloride (DAPI) nuclear stain for 30 minutes.

All images were acquired using an Olympus FV1000 Multiphoton Laser Scanning Microscope with a step depth of 5 Step counts started at the surface (step 0) and imaged 26 steps in the z-direction (125 μm depth). Three channels were imaged, including the fluorescent label, the second harmonic to visualize collagen networks and tissue structure, and DAPI.

In vivo mouse clearance studies. All animal experiments were performed in accordance with protocols approved by the Committee on Animal Care at MIT. Female, C57BL/6 mice 15 weeks old were used for this study. Animals were anesthetized using isoflurane during the treatment. A custom-made 40 kHz ultrasound probe was used to administer ultrasound locally in the colon (Sonics and Materials, Inc., Newtown, Conn.). The intensity of treatment was calibrated to 4.0 W by calorimetry. Treatment consisted of a 0.5 second burst of ultrasound.

3 kDa and 70 kDa Texas red-labeled dextrans were used as model permeants to maintain a constant permeant chemistry while isolating the effect of permeant size. A 0.5-mL enema of dextran at a concentration of 0.33 mg/mL was instilled into the colon. The ultrasound probe was immediately inserted into the colon and sonication took place for approximately 0.5 seconds. After sonication, the ultrasound probe was removed with the animal still sedated by isoflurane. The dextran solution was allowed to sit in the colon for 2 minutes. After that time, the colon was thoroughly lavaged with PBS to remove any residual dextran that did not penetrate the tissue. Groups of animals were euthanized immediately after, or 30 minutes after the excess dextran was washed out of the colon. After sacrifice, the animals' colons were dissected out. Fluorescent intensity was quantified by imaging the colons using an IVIS Fluorescent Imaging System (Perkin-Elmer, Waltham, Mass.) using the same procedure described above.

Statistical analysis. Statistical analysis was performed using one-way analysis-of-variance (ANOVA) tests with multiple comparisons unless otherwise stated. Statistical significance was defined as P <0.05. All statistical calculations were performed in MatLab R2015a.

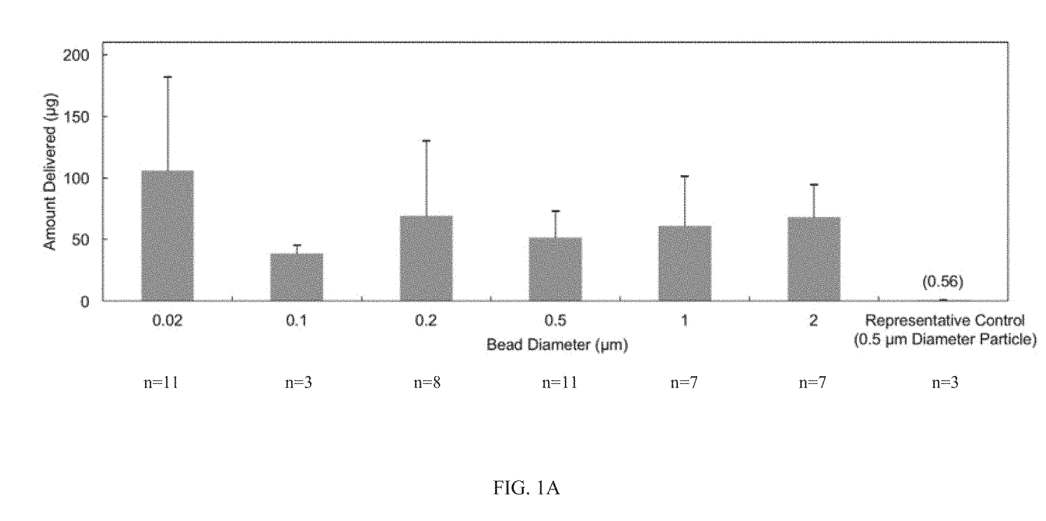

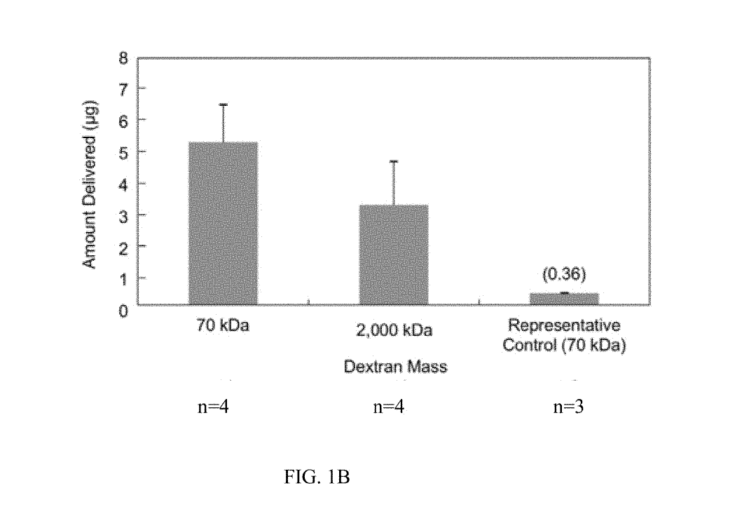

Example 1: Effect of Material Size on Delivery. The impact of permeant size on its ability to be delivered using ultrasound was investigated. It was hypothesized that larger permeants and particles would be delivered to a lesser extent because of increased steric hindrance. Latex beads with diameters spanning two orders of magnitude and dextran polymers were utilized to examine the effect of both rigid defined shapes (latex particles) and free polymer chains (dextrans). At the same time, the effect of size alone may be isolated by utilizing the same chemistries for both conformational types. Fluorescent intensity was correlated to mass of the permeant using a calibration curve. Delivery of various permeants into tissue is shown in FIGS. 1A and 1B using the permeants at a concentration of 0.2 mg/mL in the donor chamber.

Ultrasound-mediated delivery was significantly greater than delivery without ultrasound. No significant difference was found between different permeant sizes delivered using ultrasound (determined by one-way ANOVA with multiple comparisons). Despite the order-of-magnitude difference in bead diameters tested, consistent delivery was achieved using ultrasound, showing no significant dependence on permeant size. The same result was observed using dextran ranging in molecular mass from 70 kDa to 2,000 kDa. Despite testing two different conformations of materials, namely rigid, spherical particles and free polymer chains, consistent delivery was observed over a wide range of sizes. interestingly, the number of latex beads delivered into tissue was an order of magnitude greater than the amount of dextran delivered, despite the smaller size of the dextran permeants tested. This is thought to be a result of the ultrasound acting on the permeant, actively propelling it into the tissue. The importance of the effect of ultrasound acting on the permeant as opposed to the tissue has previously been noted in experiments investigating delivery using both pre-treatment of tissue with ultrasound as well as simultaneous permeant-ultrasound exposure, with the latter showing significantly greater delivery.

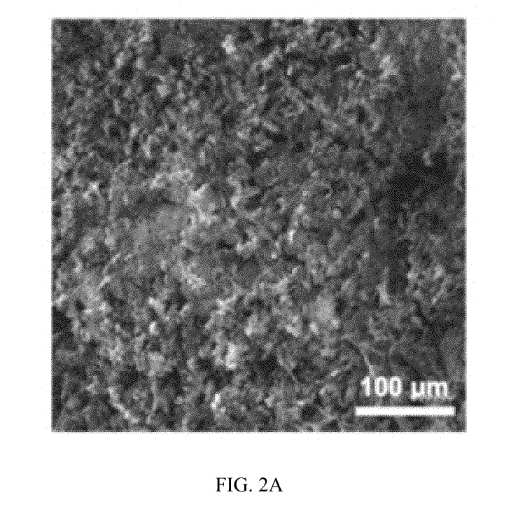

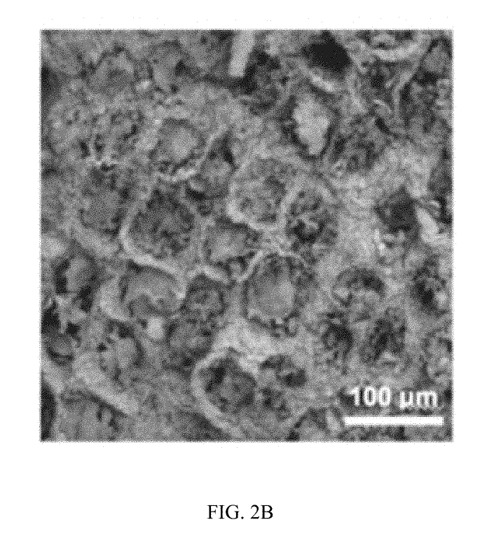

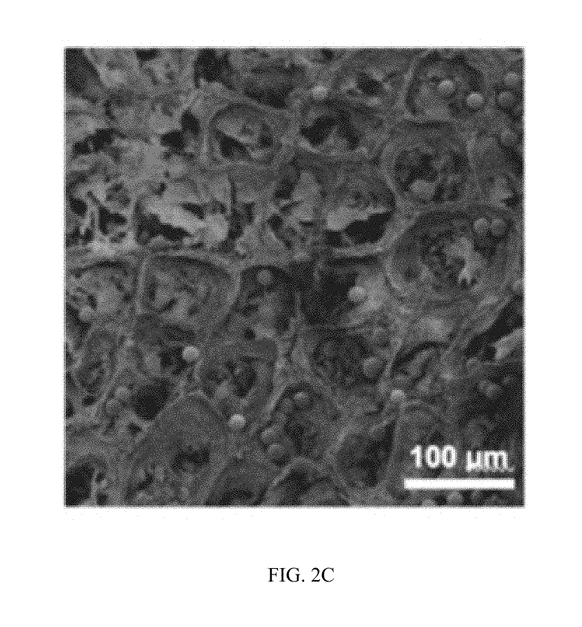

With regard to the effect of ultrasound on the tissue, SEM imaging was performed on tissue treated with ultrasound and compared to tissue not treated with ultrasound. Representative images are shown in FIGS. 2A-2C. In tissue not treated with ultrasound (FIG. 2A), crypts were not visible and were obscured by the thick mucus layer that covers GI epithelial surfaces. However, in tissue treated with ultrasound (FIG. 2B), the crypts were clearly visible and were distributed evenly.

Therefore, it seems that ultrasound acts to dissipate the mucus layer to facilitate enhanced delivery, as opposed to altering the epithelial structure. This is in agreement with other published studies, which noted negligible histological disruption to the surface colonic epithelium resulting from ultrasound treatment. Because mucus is continuously secreted, it is hypothesized that this layer would regenerate after treatment. Indeed, previous reports on chronic administration of ultrasound in the rectum have shown no deleterious effects, even in the setting of chemically-induced inflammation. The subsequent distribution of latex beads over the tissue as a result of delivery was also imaged (FIG. 2C). Treatment enabled relatively uniform distribution of the latex beads across the epithelial surface, with no clear pattern in clustering or location of the beads after delivery.

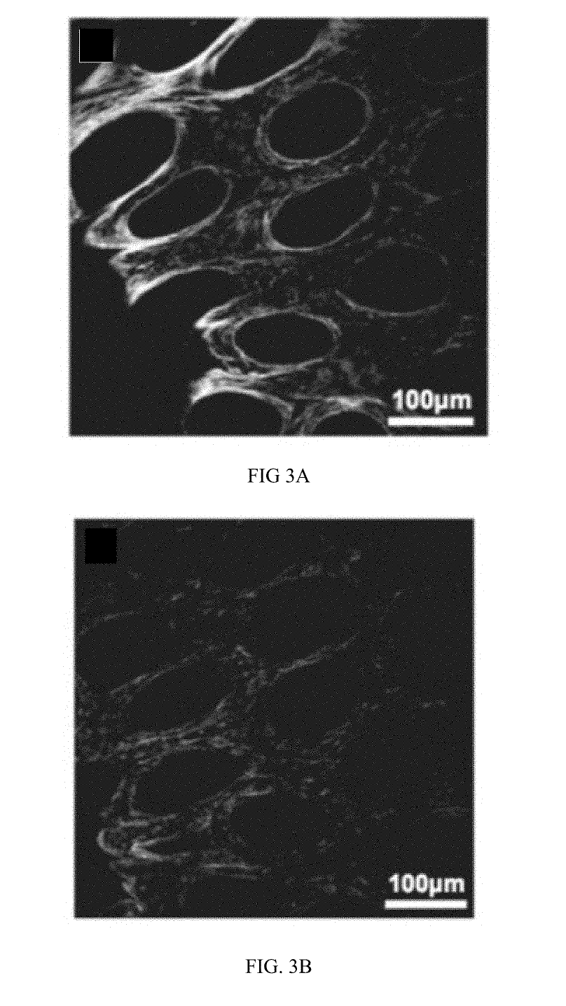

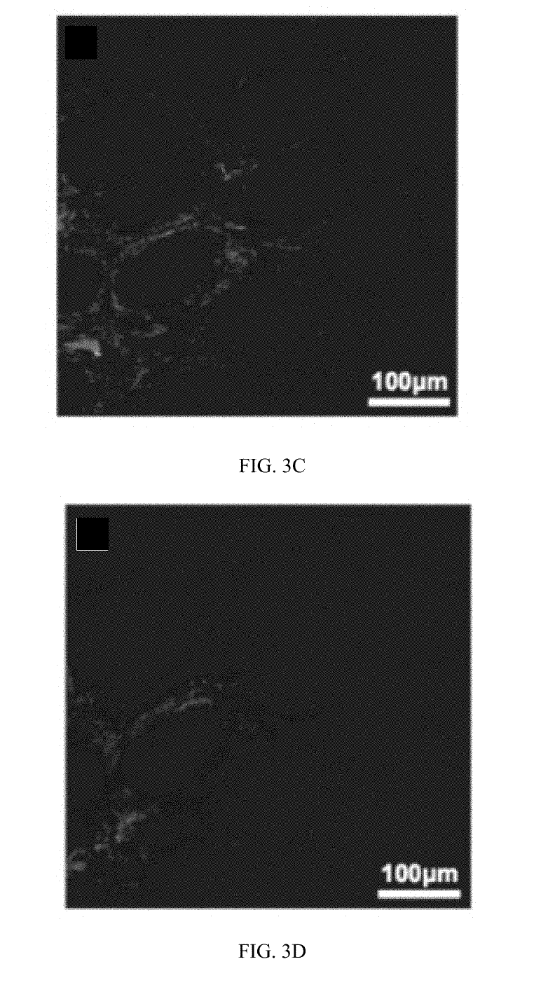

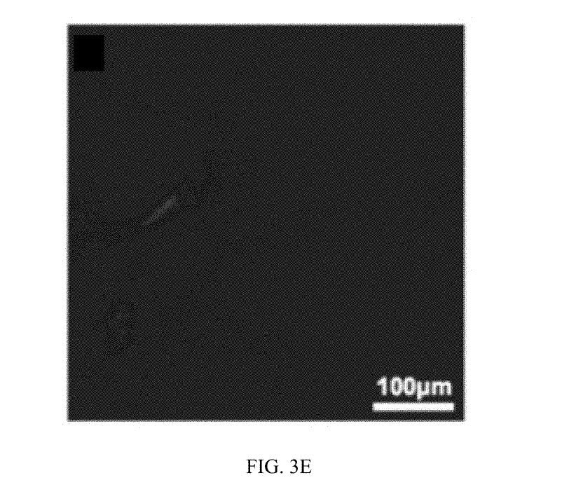

The penetration of latex beads within the tissue was also investigated using confocal microscopy. Confocal imaging was performed on porcine colonic tissue following delivery of fluorescently labeled 0.5 μm diameter carboxy late-modified latex beads and staining with DAPI. Discrete levels within the tissue are shown in FIGS. 3A-3E. Penetration into the tissue was also relatively uniform, although some clustering was observed. Interestingly, fluorescent signal was observed at depths of up to 125 μm into the tissue from the ultrasound exposed luminal surface, demonstrating significant penetration of permeants as a result of a short ultrasound treatment. The fact that relatively large particles can be delivered deep within the tissue using ultrasound could enable the development of depot systems for the extended release of therapeutics locally in the colon.

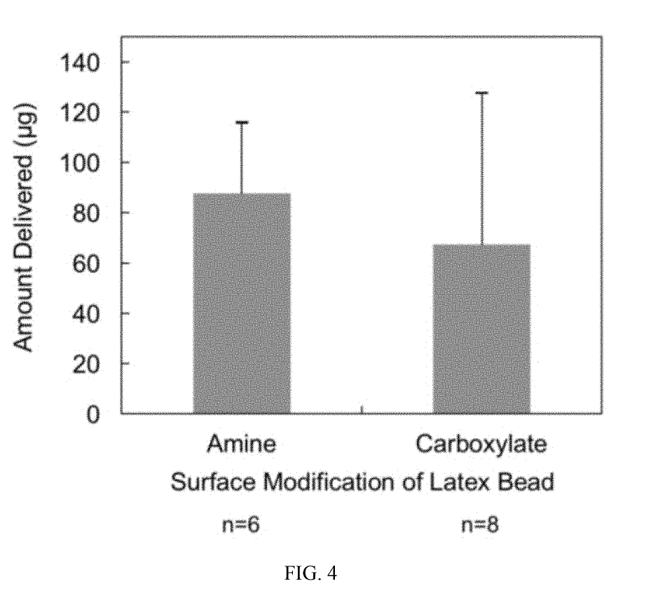

Example 2: Effect of Surface Charge on Delivery. Next, the effect of surface charge was investigated. Given the anionic nature of mucus, charge is an important parameter that is utilized in GI-based drug formulations to modulate retention and delivery. To investigate the effect of material charge on delivery, latex beads with carboxyl or amine surface modifications were utilized to impart charge on the particle. The delivery of 0.2 μm diameter beads with either amine (+0.3 atto-equivalents per particle) or carboxyl (-0.3 atto-equivalents per particle) surface modifications is shown in FIG. 4. Surface charge was found to not significantly affect the amount of material delivered into the tissue using ultrasound. This was surprising given the mucus layer is negatively charged and the epithelium is positively charged. This, again, supports the hypothesis that the predominant mechanism of ultrasound-mediated GI delivery is ultrasound acting on the permeant, as opposed to ultrasound permeabilizing the tissue directly. This will have tremendous benefit when considering the safety of this technology. If delivery is independent of charge of the material, then charge may be a parameter that could be tuned to achieve subsequent retention or preferential clearance from the tissue after ultrasound-mediated delivery.

Example 3: Effect of Treatment Time on Delivery. Given the relative insensitivity of delivery to permeant size or charge, the ultrasound treatment time utilized was varied to investigate its effect on delivery. A range of treatment times was investigated to understand in greater detail how materials interact with ultrasound. It was hypothesized that delivery would directly correlate with ultrasound treatment time.

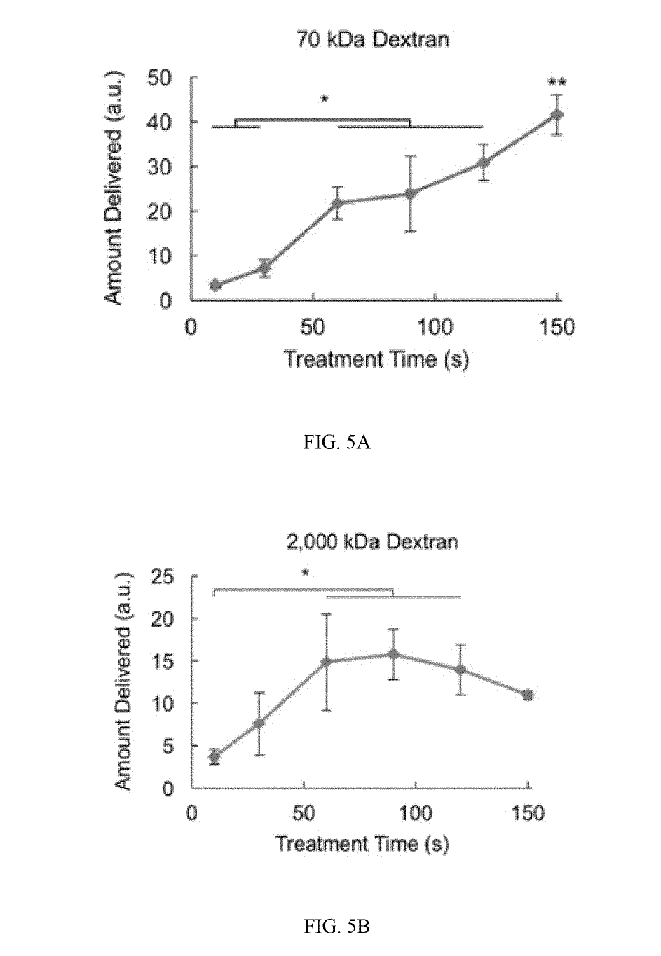

Treatment times between 10 and 150 seconds of ultrasound (20-300 seconds total permeant contact time utilizing a 50% duty cycle) were tested for three different permeants having a range of molecular masses and conformations (FIGS. 5A-5C).

Generally, the amount of permeant delivered into tissue increased with increasing ultrasound treatment time. The delivery of 70 kDa dextran correlated almost linearly with ultrasound treatment time. Interestingly, the delivery of 2,000 kDa dextran appeared to plateau with increasing ultrasound treatment time, reaching a maximum in delivery at a treatment time of 90 seconds. This result suggests that no further penetration of 2,000 kDa into tissue occurs with further ultrasound exposure. No such plateau was observed in the delivery of 70 kDa dextran or 0.5 μm diameter latex beads. If this plateau were simply a result of permeant size, then it would be expected that delivery of 0.5 μm diameter latex beads would also show a similar plateau. However, that was not the case (see FIG. 5C). Indeed, the delivery of 0.5 μm diameter latex beads increased with increasing ultrasound treatment time. Together, these findings show that the plateau in delivery of 2,000 kDa dextran is due to another material property beyond simply the permeant's size or mass. For example, the permeant conformation, in addition to overall size, may play a part in determining its deliverability and may explain why a long-chain polymer like 2,000 kDa dextran demonstrated a plateau in delivery.

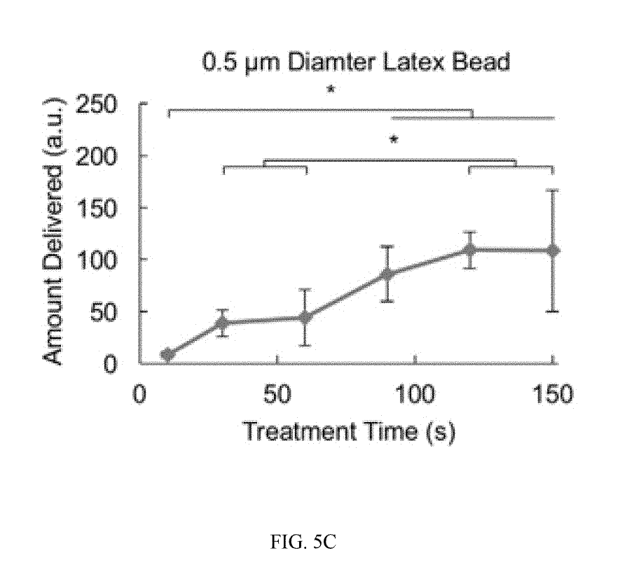

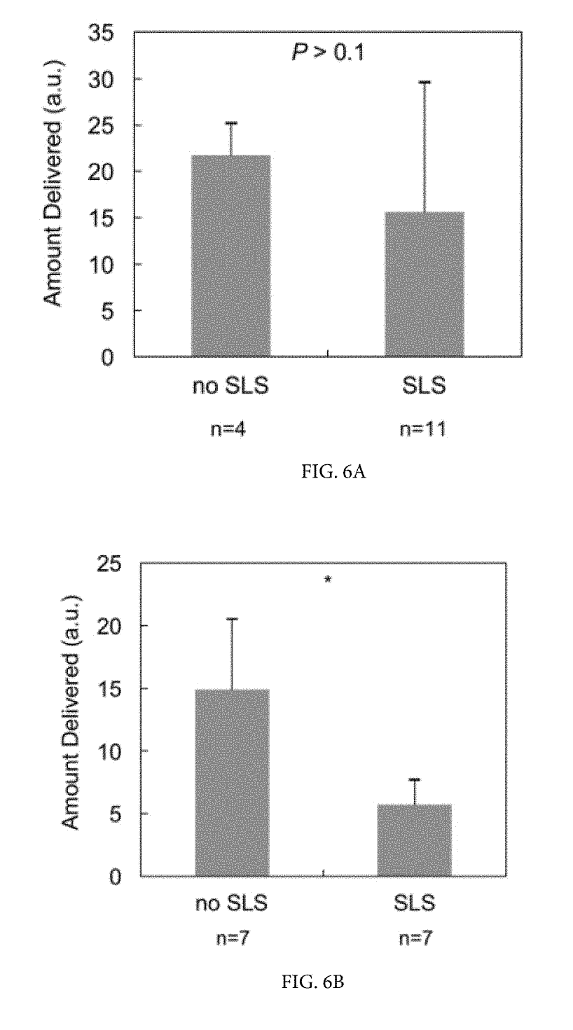

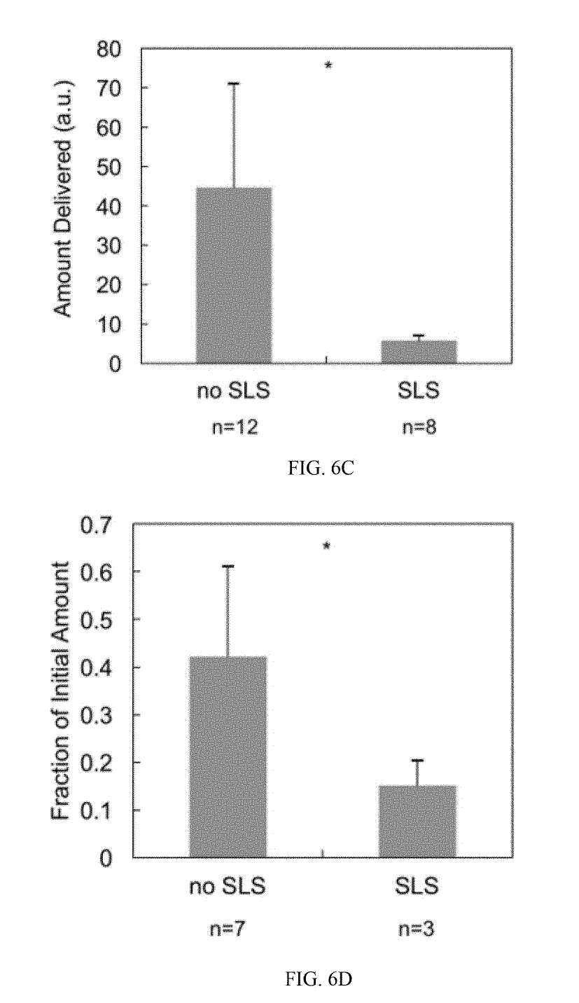

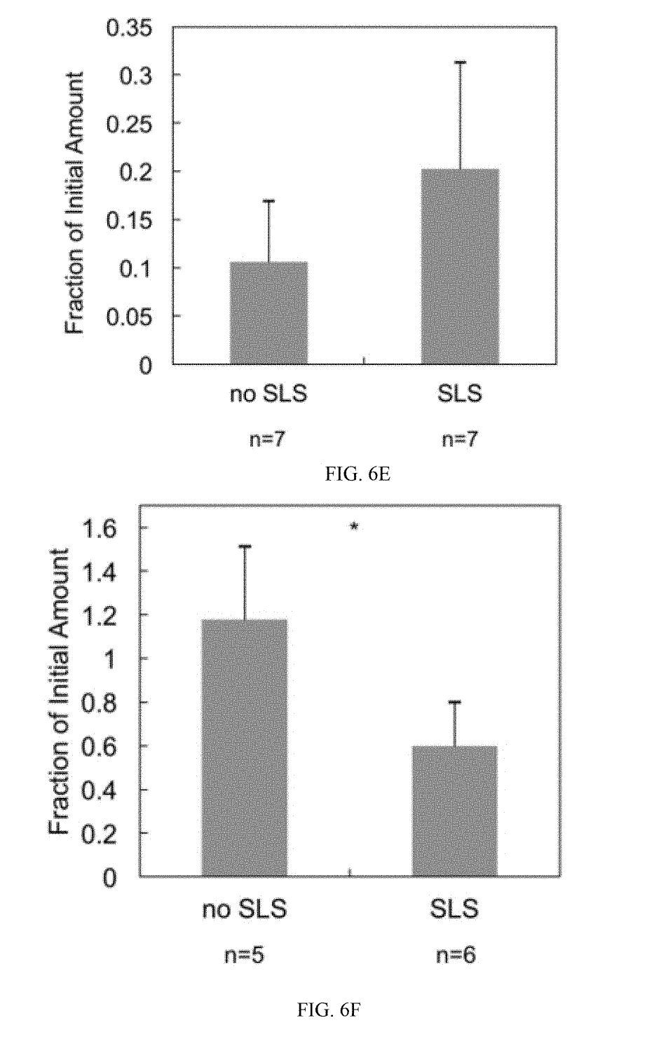

Example 4: Elect of the Simultaneous Use of Chemical Penetration Enhancers. In addition to treatment time, the use of chemical penetration enhancers (CPEs) was investigated because they have previously been shown to act synergistically with ultrasound in the context of transdermal drug delivery. However, the potential synergy of ultrasound and CPEs has not previously been investigated in the GI tract. SLS at a concentration of 1 wt % was chosen because it has been commonly employed in transdermal and oral drug delivery studies. SLS was hypothesized to further enhance delivery based on achieving an increased level of tissue permeabilization. The resulting delivery of model permeants with and without SLS is shown in FIGS. 6A-6C. It can be seen that the delivery of 2,000 kDa dextran and 0.5 μm diameter carboxylate-modified latex beads was significantly reduced by the addition of SLS to the coupling solution. The average amount of 70 kDa dextran was also reduced using SLS, however this result was not statistically significant. Multiple issues might be attributed to this result. First, there could be static repulsion effects owing to the fact that the 2,000 kDa dextran, 0.5 μm diameter carboxylate-modified latex beads, and the SLS are all negatively charged, whereas the 70 kDa dextran is zwitterionic. Another possible explanation could be the fact that SLS reduces the surface tension of the coupling solution, leading to reduced energetics of bubble collapse during transient cavitation. Given the findings presented above on the negligible role surface charge plays on delivery, it is thought that delivery is reduced upon the application of SLS because of reduced bubble collapse energetics. This would indeed result in heavier permeants being delivered less, which is what was observed in FIGS. 6A-6C.

Example 5: Permeant Clearance Tests. In addition to delivery into tissue, the subsequent clearance of the drug material can play an important role in the overall therapeutic effect. Therefore, the clearance time of model permeants from tissue was investigated after ultrasound-mediated delivery into tissue. While SLS had no effect on the immediate delivery of 70 kDa dextran, perhaps an effect would be seen at longer time scales, which would allow more time for SLS to act on the tissue to fluidize and subsequently permeabilize the barrier. As a result of increased permeability, it was hypothesized that the addition of SLS would increase the rate of clearance of materials from tissue. The results are shown in FIGS. 6D-6F.

With the exception of 2,000 kDa dextran, the addition of SLS in the donor chamber during ultrasonic treatment resulted in significantly less permeant still present in the tissue 24 hours later. The addition of SLS had no effect on the clearance of 2,000 kDa dextran. This result could be an artifact due to the resolution attainable using fluorescent probes and the inherent noise. Given the significant reduction in the delivery of 2,000 kDa dextran observed using SLS (FIG. 6B), further reductions in signal due to clearance of the 2,000 kDa dextran after 24 hours could make detecting the fluorescent signal above background noise difficult. This also explains the larger standard deviation observed in the 24-hour clearance results using SLS.

When SLS was not used during ultrasonic delivery, clearance was reduced, resulting in more permeant remaining in the tissue 24 hours after delivery. It can be seen that for both masses of dextran, approximately 80% of the initial amount of material had cleared from the tissue after 24 hours using SLS during delivery. In contrast, the 0.5 μm diameter carboxylate-modified latex beads showed significantly less clearance (FIG. 6F) when SLS was used during delivery. Even more striking, when SLS was not used, there was found to be no clearance of latex beads from the tissue 24 hours after delivery. This lack of clearance from the tissue is likely a result of the relatively large size of these particles, which would hinder their diffusion through the tissue after delivery with ultrasound. Based on this result, material size is likely to directly correlate with the residence time of the material in GI tissue and may offer an important variable for tuning novel pharmacokinetic profiles of therapeutics administered using ultrasound. This extended residence time again supports the idea that depot systems can be created to allow for extended or controlled release of drug locally in GI tissue.

Example 6: In Vivo Testing of Clearance Rate. Given the observed impact of molecular weight on subsequent clearance of permeants from the local tissue, this effect was investigated further in vivo in mice using 3 and 70 kDa dextran so as to isolate the effect of molecular size only. The two permeants were administered rectally followed by sonication using a custom-made ultrasound probe depicted in FIG. 7A. The relative amount of each permeant still present in the colonic tissue in vivo 30 minutes after delivery is shown in FIG. 7B.

From FIG. 7B, it can be seen that after 30 minutes, statistically more 3 kDa dextran had been cleared from the colon than 70 kDa dextran. Indeed, in 30 minutes, only 34% of the 70 kDa dextran had been cleared from the colon, as opposed to 86% of the 3 kDa dextran. Because the only difference between the two permeants is length of the polymer chain, the decreased rate of clearance observed in vivo for 70 kDa dextran can be attributed to its size. Based on the Stokes Radius, 70 kDa dextran has a radius approximately 2.5 times larger than the radius of 3 kDa dextran. This simple increase in molecular size has a powerful impact on subsequent clearance and serves as a proof-of-concept for tuning of the size of hypothetical drug formulations to modulate residence times in the tissue to achieve extended release.

Discussion. Ultrasound-mediated gastrointestinal delivery has the capacity to rapidly deliver a wide range of permeants with little sensitivity to the permeant itself. Short, 1-minute treatments significantly enhanced permeation and delivery of materials into epithelial tissue to depths beyond 100 μm ex vivo. This was observed irrespective of the surface charge of the permeant, which was surprising given the net negative charge of mucus. Ultrasound treatments appeared to remove the mucus layer, revealing the crypts, which explains why anionic microparticles were delivered to the same extent as cationic ones. The morphology of the permeant impacted delivery, with homogenous, spherical latex beads being delivered to a greater extent than long-chain polymers (dextran). Once delivered into tissue, permeant size was discovered to play an important role in the overall residence time in the tissue. Larger permeants are retained longer in tissue, owing to their reduced diffusion through tissue as a result of their size. This result was also confirmed in vivo in mice with 70 kDa dextran being cleared more slowly from the colon than 3 kDa dextran. This technology can be used clinically for the administration of medicated enemas, enabling the localized delivery of biologics to treat diseases such as inflammatory bowel disease. More broadly, these studies help inform further development and miniaturization of ultrasound technology to enable fully-ingestible systems for the oral delivery of complex molecules.

### Chemical Formulations, Dopants, and Methods of Enhancing Ultrasound-mediated Drug Delivery

This technology relates to the use of dopants and chemical formulations for the coupling solution to transmit an ultrasonic wave for the purposes of interacting with the wave and enhancing the delivery of a molecule also contained within the coupling solution of applied to the tissue after pre-treatment with the ultrasound and dopant or chemical formulation for applications in both the GI tract and on the skin. Further, disclosed is a method of controlling precisely the resulting permeability of tissue after ultrasound exposure using a treatment modality involving the simultaneous use of two ultrasound frequencies. This latter development is important for controlling the dose of drug delivered using ultrasound, potentially enabling the delivery of drugs that require greater control of pharmacokinetics.

Interestingly, and without wishing to be bound by any particular theory, it is believed that the method of action of the methods and compositions described herein is not a result of enhancing the permeability of tissue (the method of action claimed for traditional chemical penetration enhancers in the GI tract and skin).

Example 7: Local Drug Delivery—Rectal Enema with Ultrasound. A patient suffering from inflammatory bowel disease (i.e., ulcerative colitis or Crohn's disease) or active proctitis (inflammation of the rectum) can be treated with an ultrasound device combined with a liquid enema containing the herein disclosed chemical formulations for enhancing the delivery of a species that is also contained within the enema, or applied after subsequent ultrasound exposure, and can include, for example, a steroid, 5-aminosalicylate, an anti-inflammatory used in the treatment of ulcerative colitis, a nucleic acid, or a protein biologic with anti-inflammatory properties. Enhancement in delivery can be through the use of chemical formulations that synergize with the ultrasound, or through the use of dopants that modulate the activity, intensity, and number of transient cavitation events. Upon administration of the enema, a brief ultrasound pulse is delivered either via the same enema-administering device or a separate ultrasound-emitting device, thus augmenting the amount of drug delivered to the tissue. Although inflammatory bowel disease is often treated with enemas, these pose a significant challenge given the requirement for retention of a liquid. By further enhancing the amount of therapeutic delivered to the tissue, the required retention time of the enema is decreased, a significant improvement on the current state of the art. Furthermore, the novel chemical formulation of the enema can further augment the amount of drug delivered compared to that which reaches the tissue with the use of ultrasound alone. It has been previously recognized that higher concentrations of drugs, such as 5-aminosalicylates, in the affected tissue inversely correlate with the severity of disease. Therefore, the chemical formulation in combination with ultrasound should prove effective in lowering disease activity.

Example 8: Local Drug Delivery—Gastro-Intestinal Ultrasound Treatment. Conditions which affect a significant surface area of the gastrointestinal system can be treated through the administration of a medicated enema into the (3I tract with subsequent ultrasound administration. Examples of such conditions include gastrointestinal bleeding and the administration of anti-fibrinolytics such as tranexamic acid, neoplasia and chemotherapeutic agents, inflammatory conditions such as eosinophilic esophagitis or Celiac disease, which benefit from steroid-based treatment.

Example 9: Systemic Drug Delivery. A large surface area bathed in a medicated enema which is exposed to ultrasound can allow sufficient systemic delivery of certain drugs, including biologics, given the dramatic increase in delivery using a novel chemical formulation for the enema. This can also be through the use of other ultrasound-emitting devices, such as ingestible capsules or lollipop-like devices (FIG. 8B),

Example 10: Method for Rapid Screening of New Formulations and Therapeutics. The high-throughput setup utilized allows for multiple chemical formulations and or new therapeutic entities to be delivered and screened to a variety of tissues ex vivo to identify those showing desirable characteristics. Utilizing ultrasonic devices with multiple probe elements allows for delivery of test formulations in a well plate-like setup. One can mount tissue in the setup shown in FIGS. 9A-9C, which creates multiple, discrete diffusion chambers on a single piece of tissue ex vivo. Each discrete well can then be loaded with a different formulation, therapeutic, or permeant, to screen in a high-throughput manner those materials that show desirable properties. These properties include enhanced delivery of permeants over that achieved using ultrasound alone, successful knockdown of a target protein using a model antisense, formulations which preferentially deliver to the tissue and remain there for extended periods of time, for example.

Example 11: Modulation of Transient Cavitation Activity. The use of specific dopants has been shown to modulate and enhance transient cavitation activity, maximizing subsequent permeability of a tissue treated with ultrasound. This method can be used to enhance the delivery of a wide-range of molecules, including small molecules, proteins, biologics, or nucleic acids through either simultaneous administration of ultrasound and the molecule, or through step-wise administration.

Example 12: Precise Control of Tissue Permeability Using Ultrasound. Provided is a new method of precisely controlling the permeability of tissue achieved after ultrasound treatment. This new technology can enable tight control of resulting permeability, which directly impacts the dose of material delivered. This control is important for successful translation of this technology to the clinic and is a capability that has not been possible to date.

Example 13: Identification o Novel Chemical Formulations Using Multi-Element Diffusion System. Novel chemical formulations that can act synergistically with ultrasound were identified using the methodological setup shown in FIGS. 9A-9C. Porcine tissue was mounted in the multi-element diffusion system shown in FIGS. 9A and 9B. Various chemical formulations and dopants were added to the donor chambers and ultrasound applied. There was also a model permeant present in the donor chamber that was fluorescently labeled. After treatment, the tissue was taken out of the diffusion chamber setup, washed, and then imaged using a fluorescent imager to quantify the amount of fluorescent label in the tissue in the discrete locations corresponding to the areas exposed to individual donor chambers.

Experiments involving utilizing the setup shown in FIGS. 9A-9C were carried out using 3 kDa. Dextran labeled with Texas Red as the model permeant. First, fresh colon tissue was procured from pigs and mounted in the custom multi-element diffusion plate shown in FIGS. 9A-9C. The same technique applies to skin tissue.

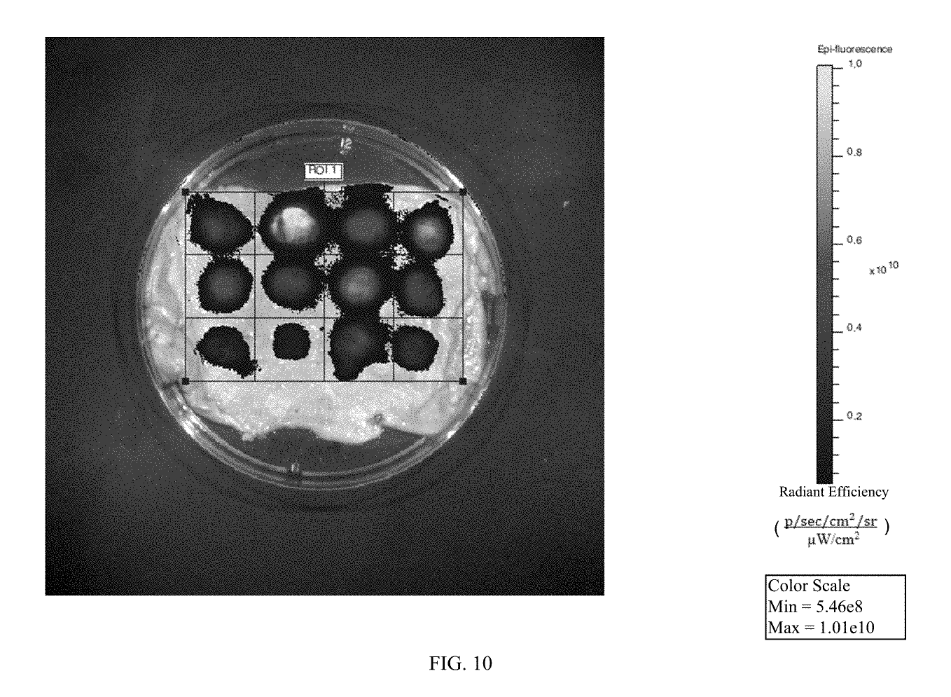

Chemical formulations were loaded into each donor chamber of the setup. Formulations were of single species at a concentration of 10 mg/mL in combination with fluorescently labeled dextran. A multi-element ultrasound horn was then used to radiate the tissue from the donor cell. Treatment was carried out for two minutes total at 50% duty cycle using 20 kHz ultrasound at 5 W/cm². After treatment, the tissue was thoroughly washed to remove any residual dextran and imaged using a fluorescent imager, resulting in a a sample image shown in FIG. 10.

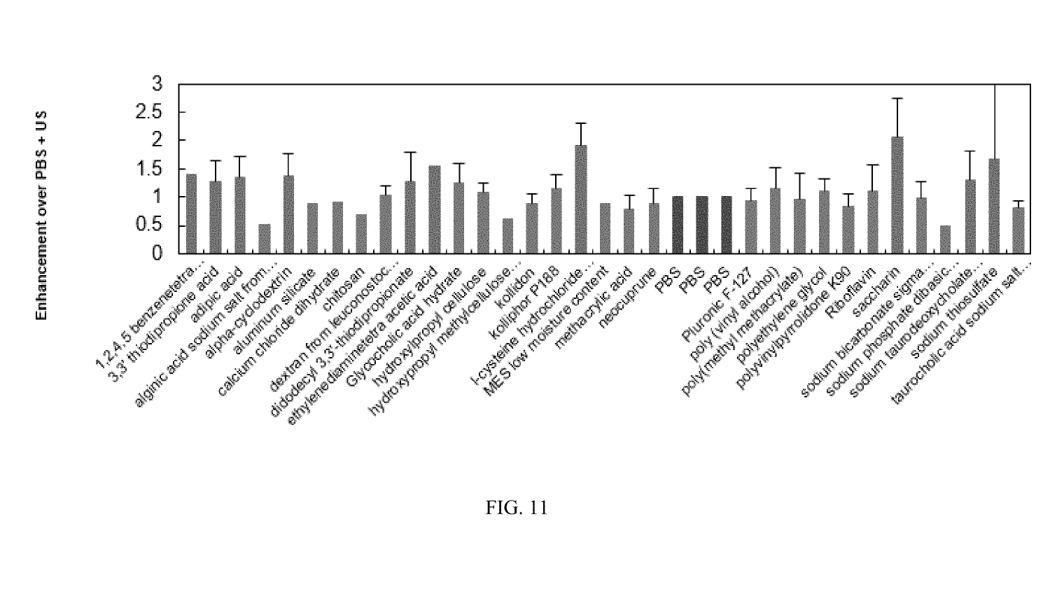

The intensity of the fluorescent signal in each discrete spot was then quantified and normalized by the intensity achieved through the use of ultrasound alone (no chemical formulation, only phosphate-buffered saline). The results of this study are shown in FIG. 11. FIG. 11 demonstrates certain chemical species that are capable of significantly enhancing the delivery of dextran. Compounds identified are listed in Table 1.

TABLE 1
Compounds from the FDA list of chemicals Generally Recognized
as Safe (GRAS) that show significant enhancement in delivery of
dextran when ultrasound is applied.

| | |
|---|---|
| 1,2,4,5 benzenetetra carboxylic acid | ethylenediaminetetra acetic acid |
| 3,3′ thiodipropione acid | l-cysteine hydrochloride |
| | monohydrate |
| adipic acid | saccharin |
| alpha-cyclodextrin | sodium taurodeoxycholate hydrate |
| didodecyl 3,3′-thiodipropionate | sodium thiosulfate |

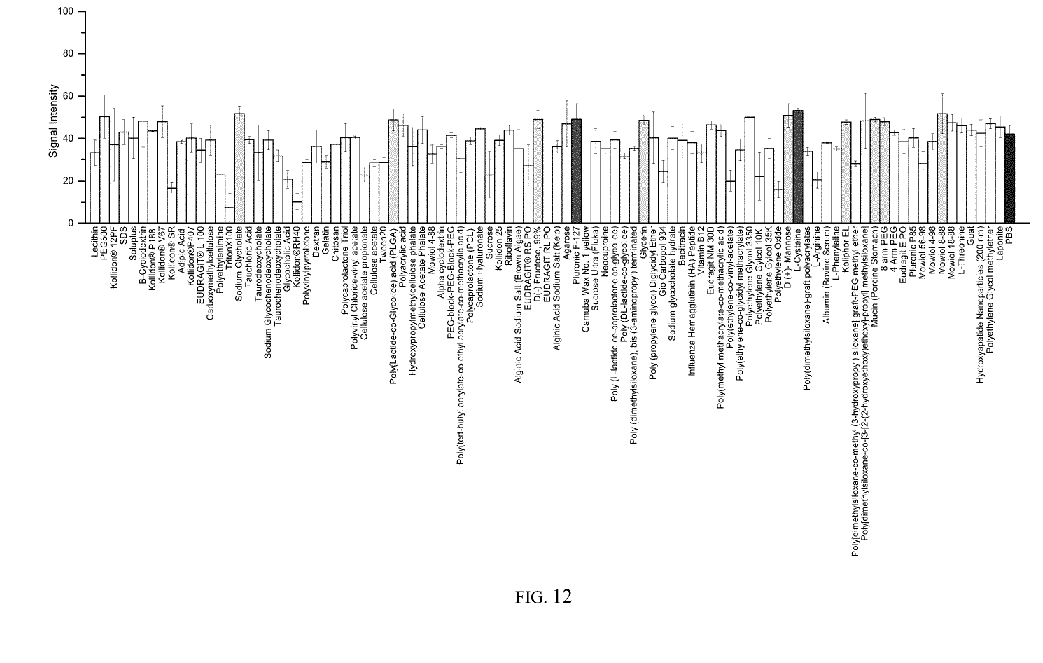

Additionally, this screening method has also been extrapolated to a 96-well system. In these tests, the model permeant was oxytocin. The results of this screen are shown in FIG. 12. Those formulations identified as providing enhancement in ultrasound-mediated delivery in a 96-well format screen are shown in Table 2.

TABLE 2
Compounds from the FDA list of chemicals Generally Recognized
as Safe (GRAS) that show significant enhancement in delivery of
oxytocin when ultrasound is applied.

| | |
|---|---|
| sodium glycholate | D(+)-mannose |
| poly(lactide glycolide) acid | kolliphor ® EL |
| D(−)fructose | mucin |
| pluronic F-127 | 8-arm PEG |
| glycerin | mowiol ® |

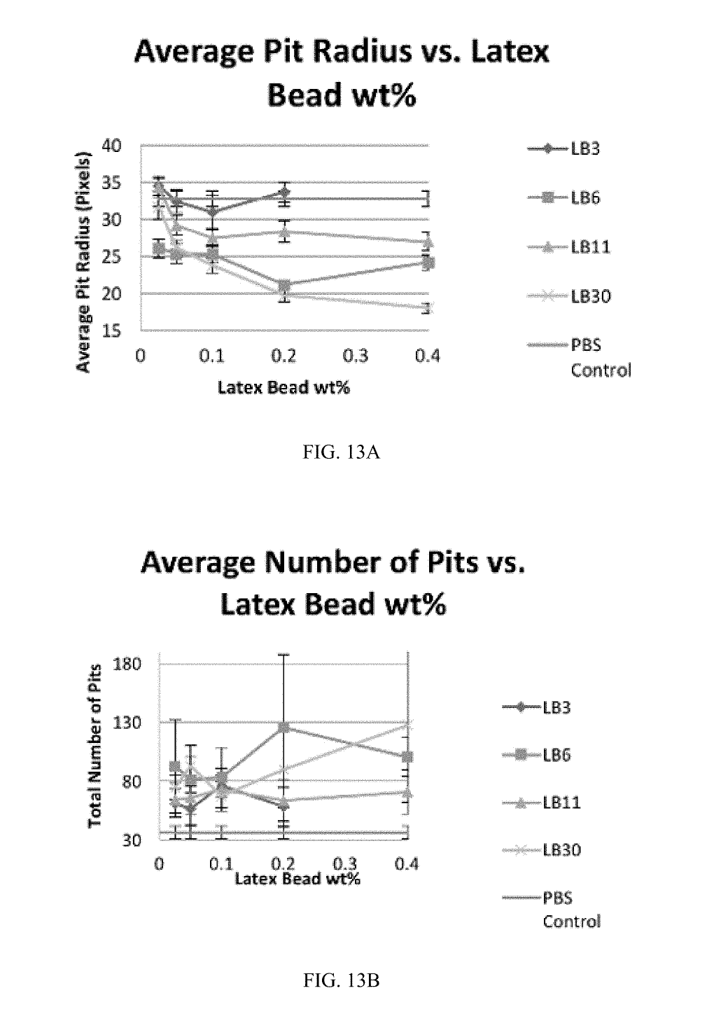

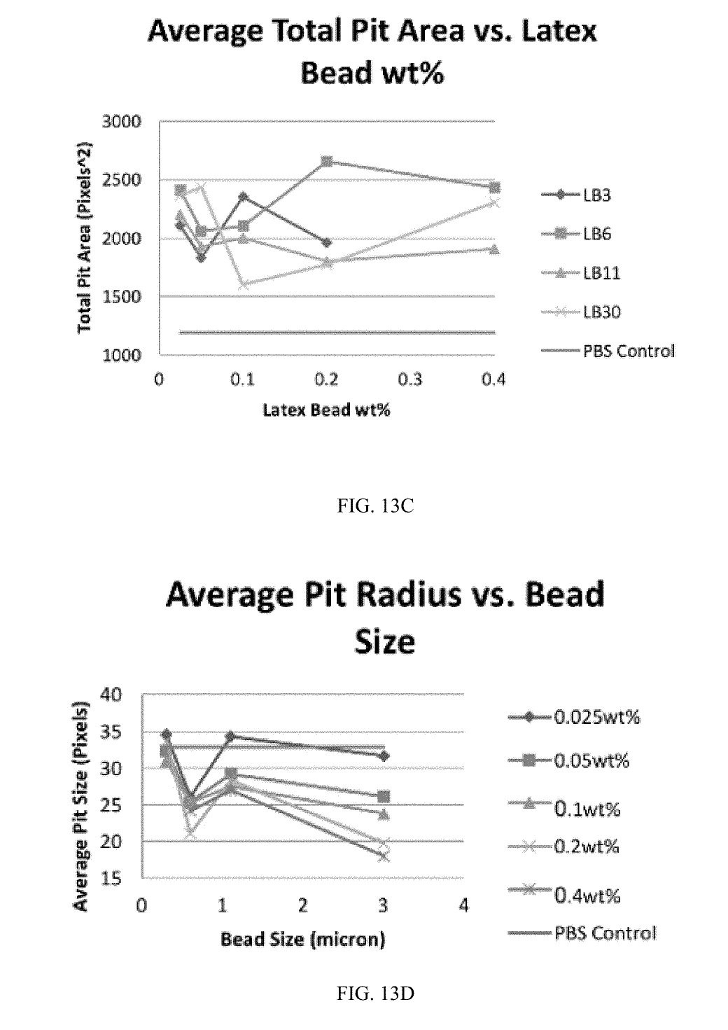

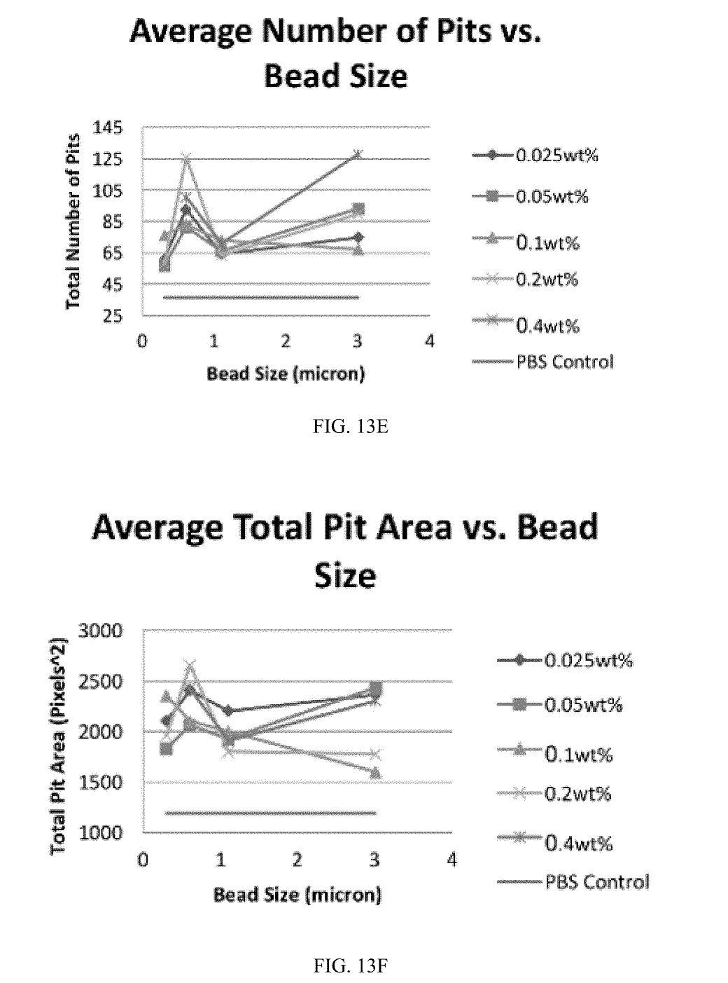

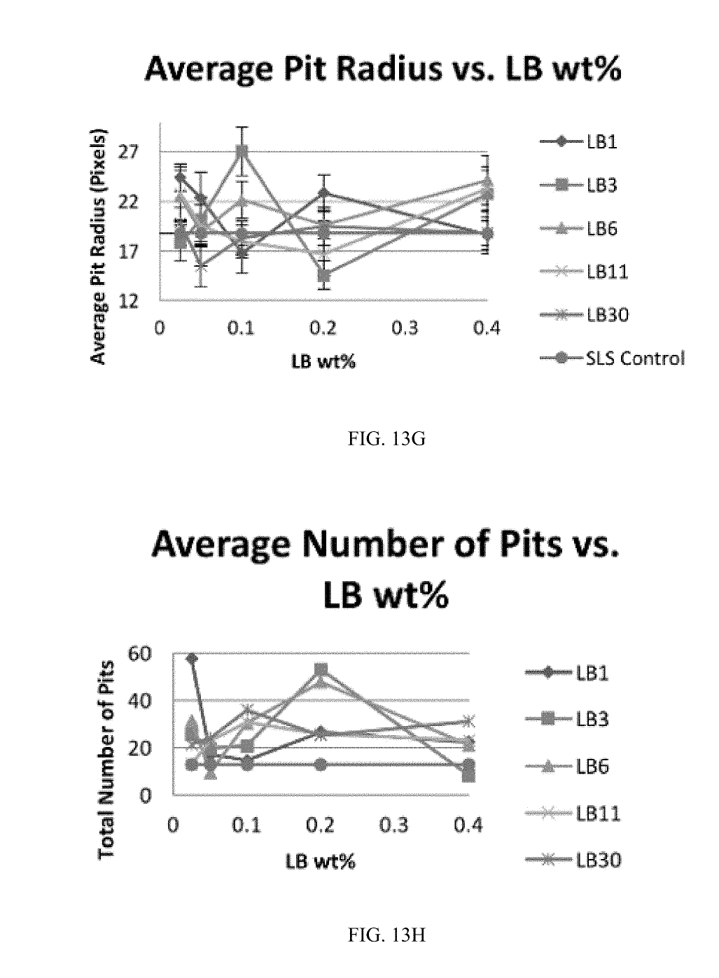

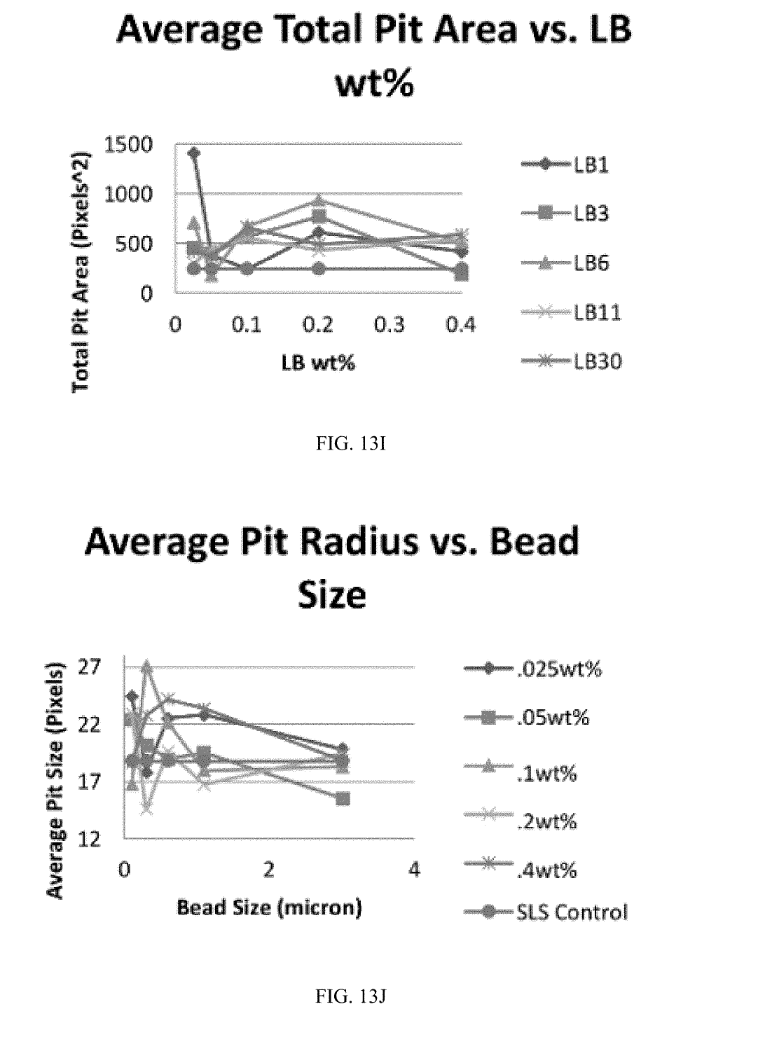

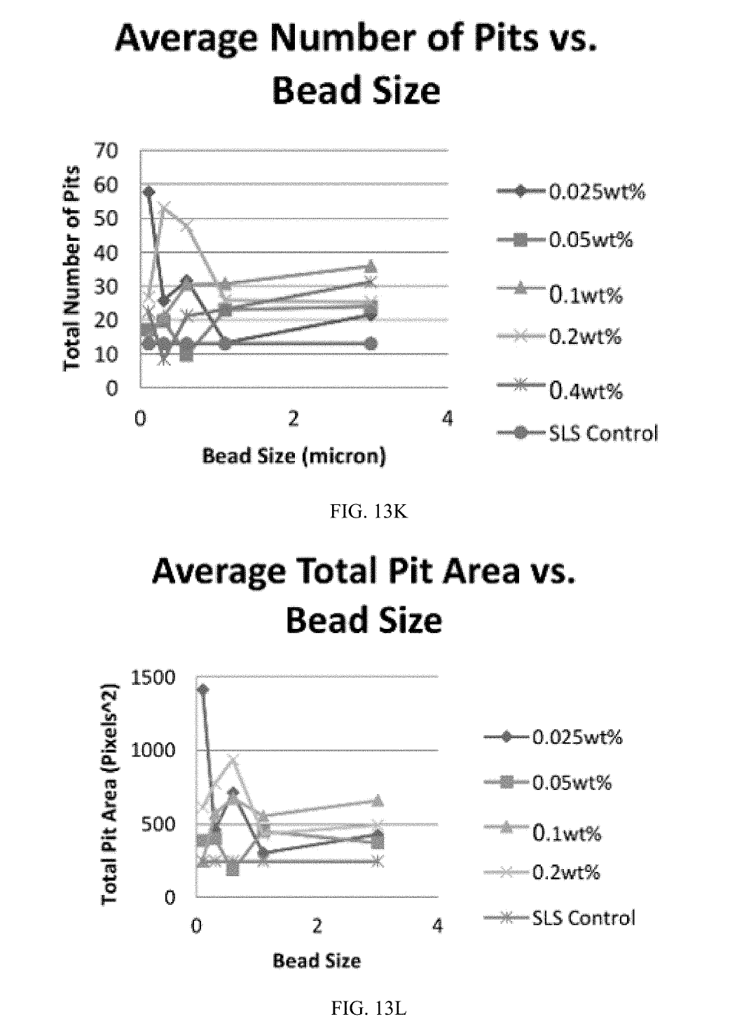

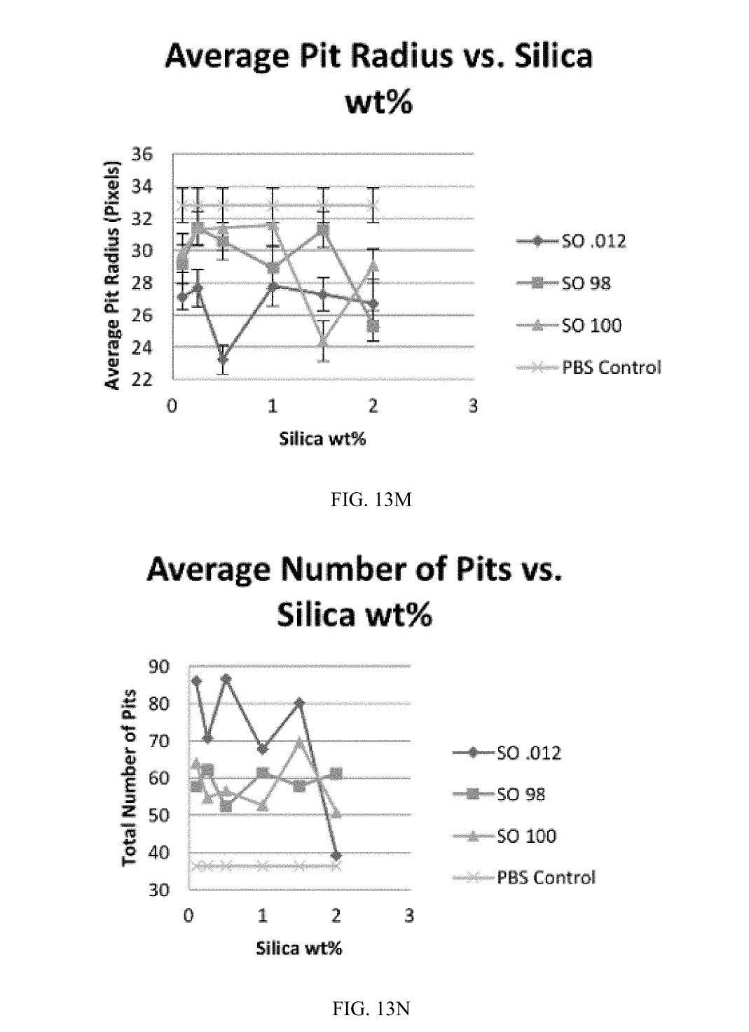

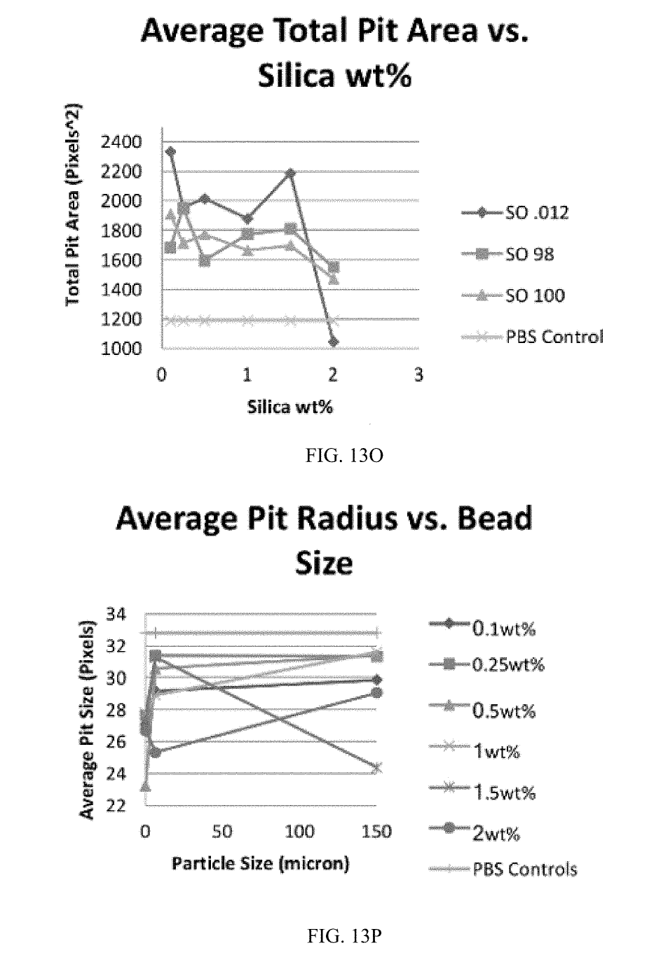

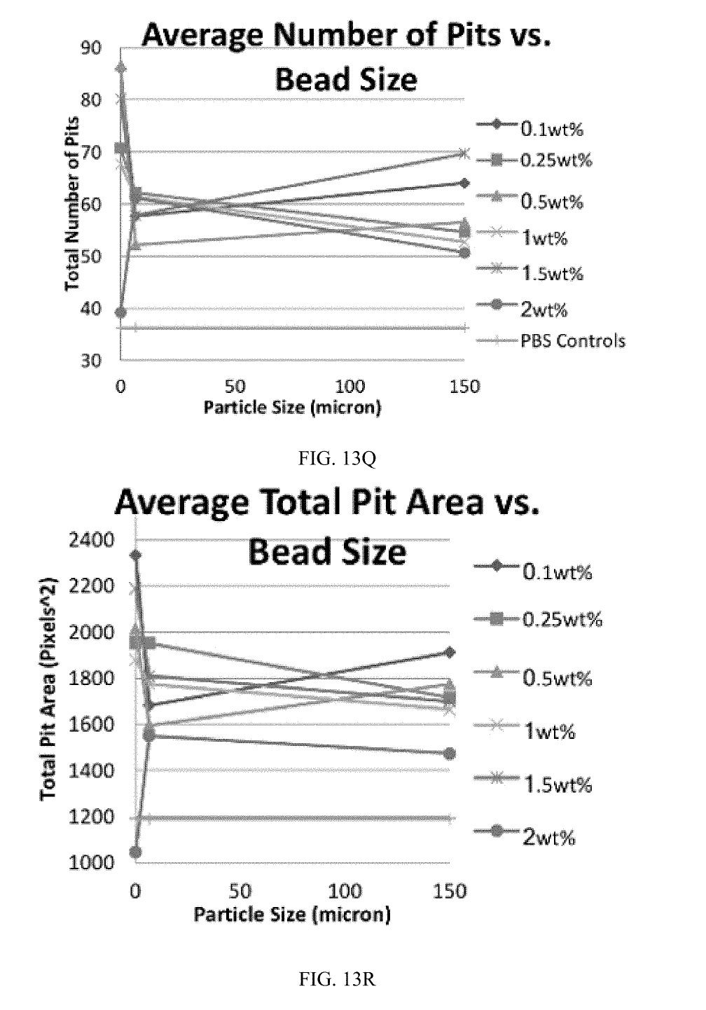

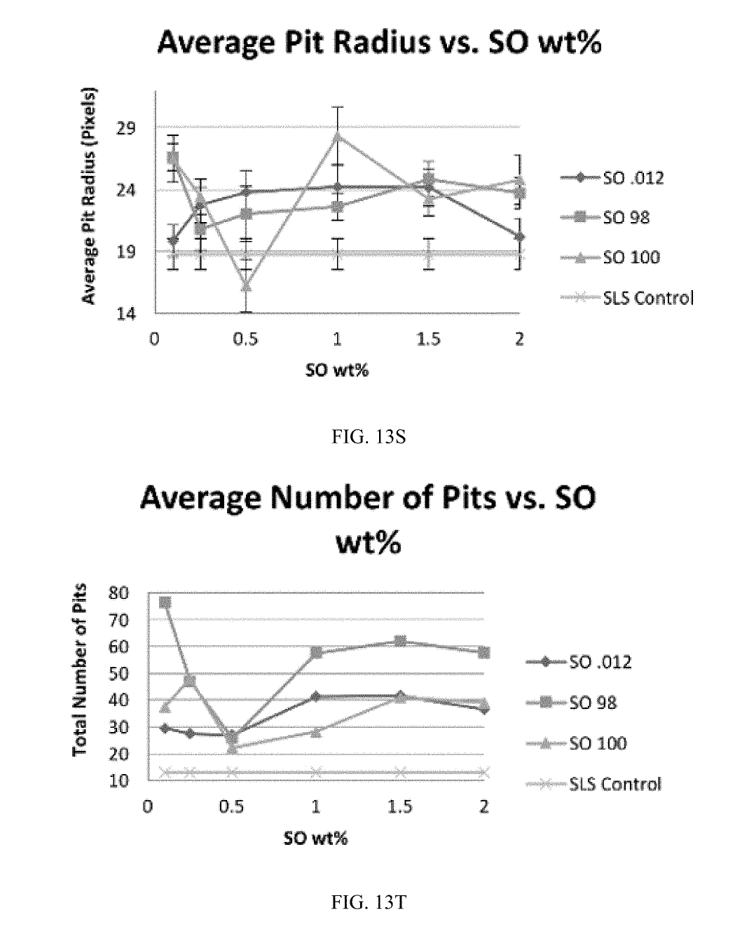

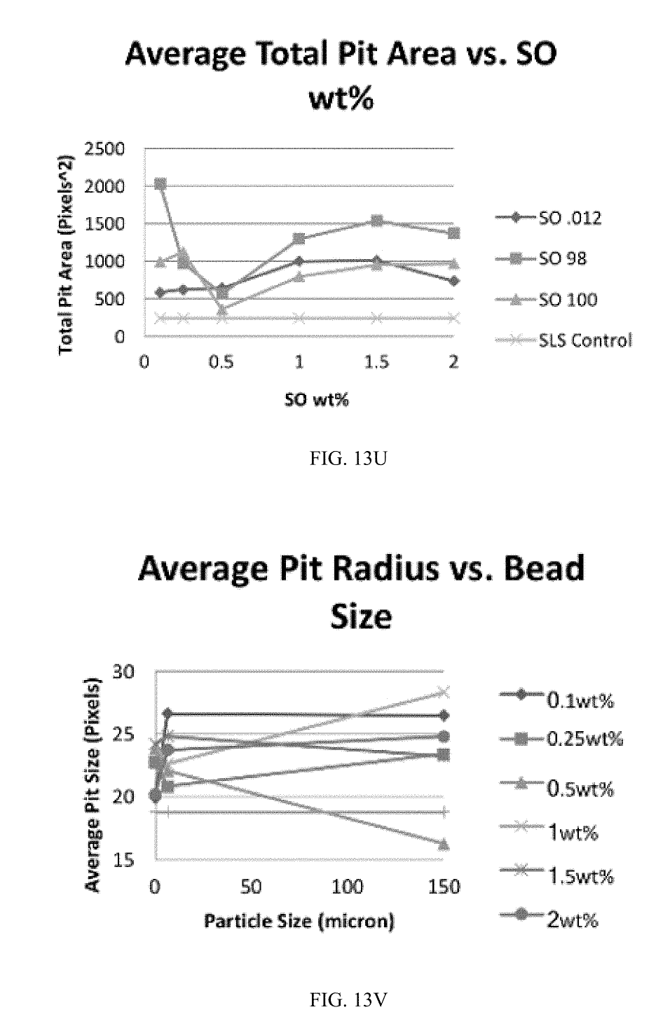

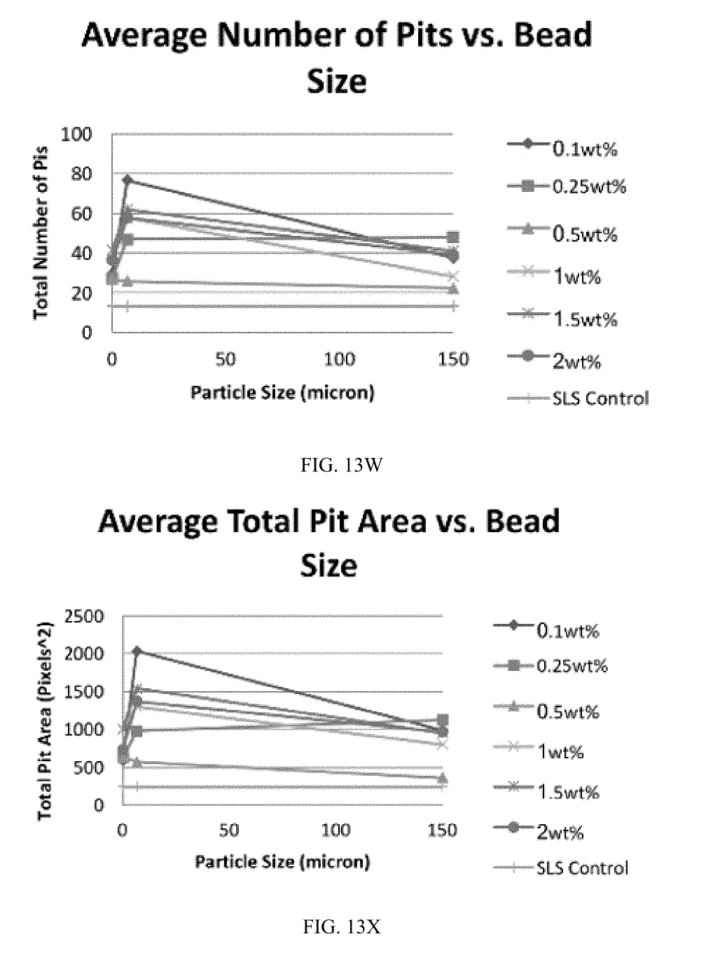

Example 14: Modulation of Transient Cavitation—Method 1: Particle Dopants. Additionally, dopants can be used to modulate the activity, intensity, or number of transient cavitation events, to enhance the permeability of tissue after treatment. Specifically, aluminum foil pitting experiments were performed. Briefly, a piece of aluminum foil was mounted in a Franz diffusion cell. Ultrasound was then applied for short bursts using a coupling solution consisting of phosphate-buffered saline (PBS) or PBS with various particles suspended throughout it. After treatment, the aluminum foil was imaged and the resulting “dents”, corresponding to transient cavitation events, were counted and the size of the dents also quantified. Both silica particles and latex beads (LBs) of different sizes were tested as dopants at different concentrations with and without 1% sodium lauryl sulfate solution in PBS (FIGS. 13A-13X).

### Observations:

### Interparticle Comparisons:

1. Effect of LB is greater than the effect of Silica in PBS
2. Effect of Silica is greater than the effect of LB in SLS Intraparticle Comparisons:

LB-SLS:
1. The presence of SLS decreases the effect of LB
2. Enhancement due to the presence of beads decreases with increasing particle size
3. Negligible dependence on particle wt % in solution over range tested here. Would generally expect there to be an optimum.
4. LB appears to increase Average number of pits compared to controls

LB-PBS:
1. There is a clear effect on particle size.
2. Negligible dependence on particle wt % in solution over range tested here. Would generally expect there to be an optimum.
3. LB appears to decrease Average pit radius compared to controls
4. LB increases Average number of pits compared to controls leading to increase in total pit area compared to controls

Silica-SLS:
1. The presence of SLS slightly decreases the effect of Silica.
2. Appears to be an optimal particle size for increasing the number of pits and overall pitted area
3. Particle wt % effect on pit radius unclear, however local minima possibly observed in number of pits.
4. Silica appears to increase all measurements compared to controls

Silica-PBS
1. Particle wt % in solution seems to slightlyecrease overall pitted area as particle wt % is increased
2. Presence of silica appears to decrease average pit size compared to controls
3. Presence of silica appears to increase average number of pits compared to controls, leading to increased total pit area compared to controls

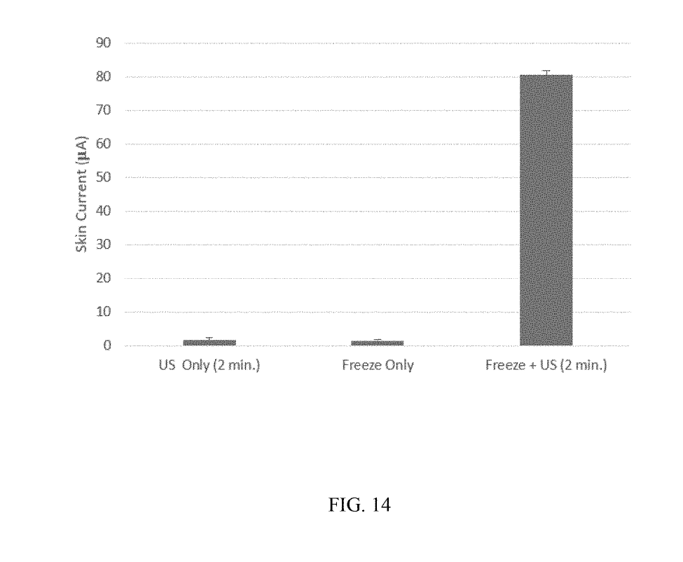

Example 15: Modulation of Transient Cavitation—Method 2: Tissue Freezing to increase Hardness. A second method to modulate cavitation events was observed through the modulation of tissue hardness by pre-freezing the tissue. Specifically, skin samples were mounted in diffusion cells. Prior to ultrasound exposure, the tissue was briefly frozen by exposing the surface to liquid nitrogen. This frozen tissue was then immediately treated with ultrasound. Electrical current measurements were recorded of the native skin, immediately after freezing (but before ultrasound exposure), and after ultrasound exposure (FIG. 14).

This method is useful for both enhancing the resulting permeability of tissue after treatment, as well as decreasing the required ultrasound treatment time significantly. Both of these features add to the clinical utility of the technology.

Example 16: Method of Precise Control of Tissue Permeability as a Result of Ultrasound Treatment. The ability to precisely control the resulting permeability of tissue after pre-treatment with ultrasound is shown. This is a capability that has to date been lacking and not possible. This capability, however, will impart significant benefit to clinical utility as permeability directly controls the dose of drug that may be delivered and the types of molecules deliverable.

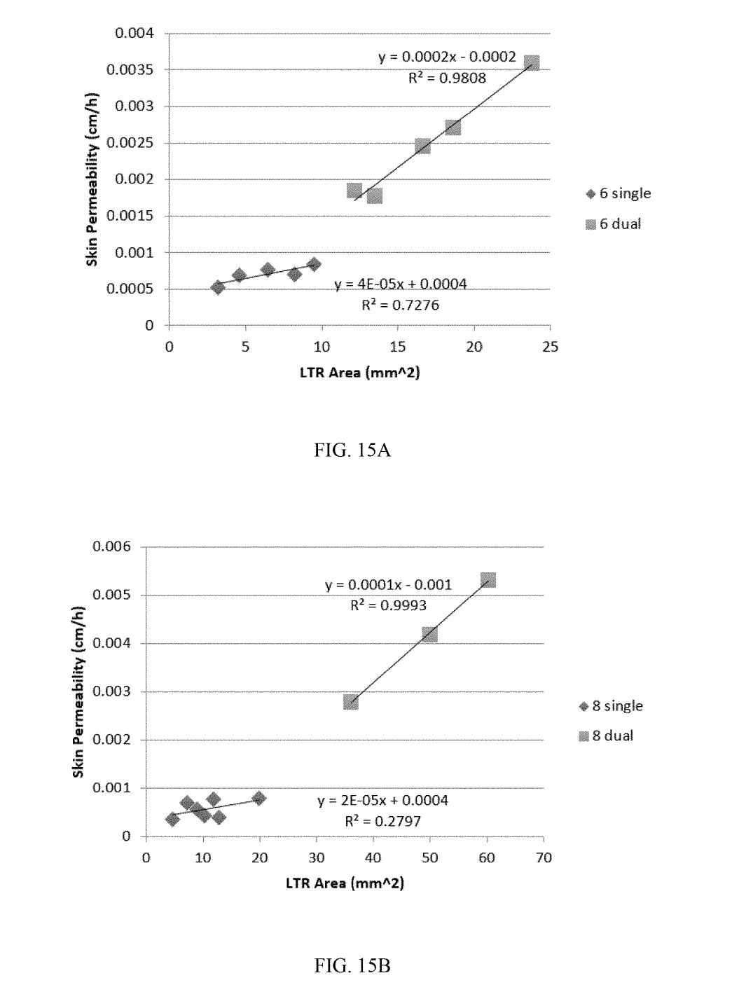

The studies on the use of dual-frequency ultrasound were extended to enable the control of permeability using only the treatment time (or a visual indicator, such as localized transport region size), as the independent variable. Porcine skin was mounted in Franz diffusion cells. Twenty-kHz ultrasound was used as the low-frequency probe and operated at a 50% duty cycle (Is on, is off) in combination with 1 MHz high-frequency ultrasound operating continuously. Additionally, 1% SLS was used in the coupling solution. After treatment, the skin was stained with a 0.04 wt % solution of allura red to detect localized transport regions (LTRs) (the areas most highly permeabilized due to the ultrasound treatment). The skin permeability (quantified using permeation of fluorescently-labeled 4 kDa dextran) as a result of LTR size is shown in FIGS. 15A and 15B for 6-minute and 8-minute ultrasound treatments, respectively.

It was shown that skin treated with low-frequency ultrasound alone in FIGS. 15A and 15B shows no correlation between LTR area and resulting permeability. This confounds the potential for clinical use since specific permabilities are desired to achieve dosing of particular drugs. Using dual-frequency ultrasound, it is shown the capacity to achieve a desired permeability without the need for any real-time measurements utilizing invasive procedures such as electrodes injected below the skin.

Example 17: Markets.

### Gastrointestinal Diseases:

Colonic inflammation—inflammatory bowel disease

Intestinal inflammation—Celiac disease, eosinophilic enteritis

Esophageal inflammation—eosinophilic esophagitis

Oral inflammation

### Urinary Tract:

Interstitial cystitis with application of chemical formulation with lidocaine during cystoscopy

### Reproductive Tract:

vaginal administration for antibiotic therapy

vaginal/cervical cancer therapy

### Intraoperative Based Therapy:

Area directed application of a drug, e.g., to the peritoneum for peritoneal metastases

### Skin

### Vaccination:

Greater permeability of the skin to allow greater quantities of larger antigen particles through the skin to the underlying cells of the immune system.

### Local Drug Delivery:

Delivery of a variety of steroids to treat dermatitis.

Enhanced delivery of anti-inflammatory agents to treat psoriatic lesions.

Delivery of salicylic acid or other irritant to the site of warts.

### Systemic Delivery:

Insulin for the control of blood-glucose levels.

Other macromolecules including proteins not currently able to be delivered.

### Cosmetic Applications:

Topical administration of skin appearance-modifying agents

### REFERENCES

[1] L. M. Ensign, R. Cone, and J. Hanes, “Oral drug delivery with polymeric nanoparticles: The gastrointestinal mucus barriers,” Advanced drug delivery reviews, vol. 64, no. 6, pp. 557-570, 2012.
[2] S. Mitragotri, P. A. Burke, and R. Langer, “Overcoming the challenges in administering biopharmaceuticals: formulation and delivery strategies,” Nat Rev Drug Discov, vol. 13, no. 9, pp. 655-672, August 2014.
[3] K. Whitehead, N. Karr, and S. Mitragotri, “Safe and Effective Permeation Enhancers for Oral Drug Delivery,” Pharm Res, vol. 25, no. 8, pp. 1782-1788, 2008.
[4] Y.-A. L. Lee, S. Zhang, J. Lin, R. Langer, R. and G. Traverso, “A Janus Mucoadhesive and Omniphobic Device for Gastrointestinal Retention,” Adv. Healthcare Mater., vol. 5, no. 10, pp. 1141-1146, May 2016.
[5] R. A. Cone, “Barrier properties of mucus,” Advanced drug delivery reviews, vol. 61, no. 2, pp. 75-85, February 2009.
[6] L. Liu, W. Yao, Y. Rao, X. Lu, and J. Gao, “pH-Responsive carriers for oral drug delivery: challenges and opportunities of current platforms.,” Drug Deliv, vol. 24, no. 1, pp. 569-581, November 2017.
[7] M. Goldberg and I. Gomez-Orellana, “Challenges for the oral delivery of macromolecules,” Nature Reviews Drug Discovery, vol. 2, no. 4, pp. 9-295, April 2003.
[8] D. S. Wilson, G. Dalmasso, L. Wang, S. V. Sitaraman, D. Merlin, and N. Murthy, “Orally delivered thioketal nanoparticles loaded with TNF-a-siRNA target inflammation and inhibit gene expression in the intestines,” Nature Materials, vol. 9, no. 11, pp. 923-928, October 2010.
[9] H. Ali, B. Weigmann, M. F. Neurath, E. M. Collnot, M. Windbergs, and C. M. Lehr, “Budesonide loaded nanoparticles with pH-sensitive coating for improved mucosa(targeting in mouse models of inflammatory bowel diseases,” Journal of Controlled Release, vol. 183, pp. 167-177.
[10] T. Gajanayake, R. Olariu, F. M. Leclerc, A. Dhayani, Z. Yang, A. K. Bongoni, Y. Banz, M. A. Constantinescu, J. M. Karp, P. K. Vemula, R. Rieben, and E. Vogelin, “A single localized dose of enzyme-responsive hydrogel improves long-term survival of a vascularized composite allograft,” Science Translational Medicine, vol. 6, no. 249, pp. 249ra110-249ra1 10, August 2014.
[11] K. Sonaje, K.-J. Lin, J.-J. Wang, F.-L. Mi, C.-T. Chen, J.-H. Juang, and H.-W. Sung, “Self-Assembled pH-Sensitive Nanoparticles: A Platform for Oral Delivery of Protein Drugs,” Adv. Funct. Mater., vol. 20, no, 21, pp. 3695-3700, October 2010.
[12] Y. Jin, Y. Song, X. Zhu, D. Zhou, C. Chen, Z. Zhang, and Y. Huang, “Goblet cell-targeting nanoparticles for oral insulin delivery and the influence of mucus on insulin transport.,” Biomaterials, vol. 33, no. 5, pp. 1573-1582, February 2012.
[13] A. Frede, B. Neuhaus, R. Klopfleisch, C. Walker, J. Buer, W. Muller, M. Epple, and A. M. Westendorf, “Colonic gene silencing using siRNA-loaded calcium phosphate/PLGA nanoparticles ameliorates intestinal inflammation in vivo,” J Control Release, vol. 222, pp. 86-96.
[14] E. M. Pridgen, F. Alexis, T. T. Kuo, E. Levy-Nissenbaum, R. Karnik, R. S. Blumberg, R. Langer, and O. C. Farokhzad, “Transepithelial Transport of Fc-Targeted Nanoparticles by the Neonatal Fc Receptor for Oral Delivery,” Science Translational Medicine, vol. 5, no. 213, pp. 213ra1 67-213ra167, November 2013.
[15] A. Banerjee, J. Qi, R. Gogoi, J. Wong, and S. Mitragotri, “Role of nanoparticle size, shape and surface chemistry in oral drug delivery.,” Journal of Controlled Release, vol. 238, pp. 176-185, September 2016.
[16] C. M. Schoellhammer and. G. Traverso, “Low-frequency ultrasound for drug delivery in the gastrointestinal tract,” Expert Opin. Drug Deliv., vol. 13, no. 8, pp. 1045-1048, August 2016.
[17] C. M. Schoellhammer, A. Schroeder, R. Maa., G. Y. Lauwers, A. Swiston, M. Zervas, R. Barman, A. M. DiCiccio, W. R. Brugge, D. G. Anderson, D. Blankschtein, R. Langer, and G. Traverso, “Ultrasound-mediated gastrointestinal drug delivery,” Science Translational Medicine, vol. 7, no. 310, pp. 310ra168-310ra168, October 2015.
[18] C. M. Schoellhammer, R. Langer, and G. Traverso, “Of microneedles and ultrasound: Physical modes of gastrointestinal macromolecule delivery,” Tissue Barriers, vol. 4, no. 2, p. e1150235, April 2016.
[19] C. M. Schoellharnmer, G. Y. Lauwers, J. A. Goettel, M. A. Oberli, C. Cleveland, J. Y. Park, D. Minahan, Y. Chen, D. G. Anderson, A. Jaklenec, S. B. Snapper, R. Langer, and G. Traverso, “Ultrasound-Mediated Delivery of RNA to Colonic Mucosa of Live Mice,” Gastroenterology, vol. 152, no, 5, pp. 1151-1160, January 2017.
[20] B. E. Polat, D. Hart, R. Langer, and D. Blankschtein, “Ultrasound-mediated transdermal drug delivery: Mechanisms, scope, and emerging trends,” Journal of Controlled Release, vol. 152, no. 3, pp. 330-348, June 2011.
[21] D. Miller, N. Smith, M. Bailey, U. Czarnota., K. Hynynen, I. Makin, American Institute of Ultrasound in Medicine Bioeffects Committee, “Overview of Therapeutic Ultrasound Applications and Safety Considerations,” Journal of ultrasound in medicine: official journal of the American Institute of Ultrasound in Medicine, vol. 31, no. 4, pp. 623-634, April 2012.
[22] M. R. Prausnitz and R. Langer, “Transdermal drug delivery,” Nat Biotechnol, vol. 26, no. 11, pp. 1261-1268, November 2008.
[23] C. C. Coussios and R. A. Roy, “Applications of Acoustics and Cavitation to Noninvasive Therapy and Drug Delivery,” Annu. Rev. Fluid Mech., vol. 40, no. 1, pp. 395-420, January 2008.
[24] A. Eller, “Rectified Diffusion during Nonlinear Pulsations of Cavitation Bubbles,” J. Acoust. Soc. Am., vol. 37, no. 3, pp. 493-503, 1965.
[25] B. E. Polat, W. M. Deen, R. Langer, and D. Blankschtein, “A physical mechanism to explain the delivery of chemical penetration enhancers into skin during transdermal sonophoresis—Insight into the observed synergism,” Journal of Controlled Release, vol. 158, no. 2, pp. 250-260, March 2012.
[26] S. Mitragotri, D. Blankschtein, and R. Langer, “Ultrasound-mediated transdermal protein delivery.,” Science, vol. 269, no. 5225, pp. 850-853, August 1995.
[27] S. Danese and C. Fiocchi, “Ulcerative Colitis,” N Engl. J Med, vol. 365, no. 18, pp. 1713-1725, 2011.
[28] E.-J. Park, J. Werner, J. Beebe, S. Chan, and N. B. Smith, “Noninvasive Ultrasonic Glucose Sensing with Large Pigs (˜200 Pounds) Using a Lightweight Cymbal Transducer Array and Biosensors,” Journal of Diabetes Science and Technology, vol. 3, no. 3, pp. 517-523, April 2009,
[29] C. R. Bawiec, Y. Sunny, A. T. Nguyen, J. A. Samuels, M. S. Weingarten, L. A. Zuhkov, and P. A. Lewin, “Finite element static displacement optimization of 20-100 kHz flexural transducers for fully portable ultrasound applicator,” Ultrasonics, vol. 53, no. 2, pp. 511-517, 2013.
[30] C. M. Schoellhammer, B. E. Polat, J. Mendenhall, R. Maa, B. Jones, D. P. Hart, R. Langer, and D. Blankschtein, “Rapid skin permeabilization by the simultaneous application of dual-frequency, high-intensity ultrasound,” Journal of Controlled Release, vol. 163, no. 2, pp. 154-160, 2012.
[31] B. E. Polat, J. E. Seto, D. Blankschtein, and R. Langer, “Application of the Aqueous Porous Pathway Model to Quantify the Effect of Sodium 1eaury⁻1 Sulfate on Ultrasound-Induced Skin Structural Perturbation,” Journal of Pharmaceutical Sciences, vol. 100, no. 4, pp. 1387-1397, April 2011.
[32] T. T. Kararli, “Comparison of the gastrointestinal anatomy, physiology, and biochemistry of humans and commonly used laboratory animals,” Biopharm. Drug Dispos., vol. 16, no. 5, pp. 351-380, July 1995.
[33] C. Atuma, V. Strugala, A. Allen, and U Holm, “The adherent gastrointestinal mucus gel layer: thickness and physical state in vivo,” Am J Physiol Gastrointest Liver Physiol, vol. 280, no. 5, p. G922, May 2001.
[34] M. E. Johansson, H. Sjovall, and G. C. Hansson, “The gastrointestinal mucus system in health and disease,” Nature Reviews Gastroenterology & Hepatology, vol. 10, no. 6, pp. 352-361, June 2013.
[35] S. Hua, E. Marks, J. J. Schneider, and S. Keely, “Advances in oral nano-delivery systems for colon targeted drug delivery in inflammatory bowel disease: Selective targeting to diseased versus healthy tissue,” Nanomedicine: Nanotechnology, Biology and Medicine, vol. 11, no. 5, pp. 1117-1132.
[36] J. Kushner, D. Blankschtein, and R. Langer, “Evaluation of the porosity, the tortuosity, and the hindrance factor for the transdermal delivery of hydrophilic permeants in the context of the aqueous pore pathway hypothesis using dual-radiolabeled permeability experiments,” Journal of Pharmaceutical Sciences, vol. 96, no. 12, pp. 3263-3282, 2007.
[37] W. Yuan, Y. Lv, M. Zeng, and B. M. Fu, “Non-invasive measurement of solute permeability in cerebral microvessels of the rat,” Microvasc Res, vol, 77, no. 2, pp. 166-173 March 2009.

All ranges described herein include all integers and subranges therein.

While example embodiments have been particularly shown and described, it will be understood by those skilled in the art that various changes in form and details may be made therein without departing from the scope of the embodiments encompassed by the appended claims.

What is claimed is:

1. A composition, comprising a pharmaceutical agent and an ultrasound enhancing agent, wherein the ultrasound enhancing agent is:
an excipient at a concentration of at least about 1 mg/mL; or
a dopant at a concentration of at least about 0.05% weight/volume.

2. The composition of claim 1, wherein the pharmaceutical agent is a therapeutic agent.

3. The composition of claim 1, wherein the pharmaceutical agent is a diagnostic agent.

4. The composition of claim 1, wherein the ultrasound enhancing agent is a disulfide bond-forming agent, a ligand, a gelating agent, an anion-responsive material, an alcohol dialkyl diester, a dicarboxylic acid, a polysaccharide, a lipidopreservative, a sweetener, a bile acid or a dopant.

5. The composition of claim 1, wherein the ultrasound enhancing agent is an excipient.

6. The composition of claim 5, wherein the ultrasound enhancing agent is a disulfide bond-forming agent.

7. The composition of claim 1, wherein the ultrasound enhancing agent is a dopant.

8. The composition of claim 7, wherein the dopant is silica, latex beads or polystyrene microspheres.

9-17. (canceled).

18. The composition of claim 1, wherein the composition is a fluid.

19. A method of delivering a pharmaceutical agent to a subject in need thereof, comprising:
administering a composition of claim 1 to a region of the subject; and
delivering ultrasound to the region, thereby delivering the pharmaceutical agent to the subject.

20-21. (canceled).

22. The method of claim 1, wherein the region is the subject's skin, or a portion thereof.

23. The method of claim 1, wherein the region is the subject's gastrointestinal tract, or a portion thereof.

24. A method of delivering a pharmaceutical agent to tissue of a subject in need thereof, comprising:
administering a fluid composition of claim 18 to the subject; and
delivering ultrasound to the fluid, thereby delivering the pharmaceutical agent to the tissue of the subject.

25-26. (canceled).

27. The method of claim 24, wherein the tissue is skin, or a portion thereof.

28. The method of claim 24, wherein the tissue is gastrointestinal tissue, or a portion thereof.

29. The method of claim 19 wherein the composition is administered orally, topically, locally or a combination thereof.

30-35. (canceled).

36. The method of claim 19, wherein a plurality of frequencies of ultrasound are delivered to the region.

37-39. (canceled).

40. The method of claim 19, wherein the subject has inflammatory bowel disease or proctitis.

41-42. (canceled).

43. A method of delivering a pharmaceutical agent to tissue of a subject in need thereof, comprising:
administering a pharmaceutical agent and an ultrasound enhancing agent in one or more fluids to the subject; and
delivering ultrasound to the one or more fluids, thereby delivering the pharmaceutical agent to the tissue of the subject.

44. (canceled).

45. A method of obtaining a biological sample from a subject, comprising:
delivering a plurality of frequencies of ultrasound to a region, tissue or a portion of tissue of the subject; and
extracting the biological sample from the region, the tissue or the portion of the tissue,
thereby obtaining a biological sample from the subject.

46-51. (canceled).
